SEQUENCE LISTING (22 sequences)

SEQ ID NO: 1 - DNA, 1380 bp, Unknown
  gtfC gene
atgagtaatttattttcttcacaaacgaaccttgcatctgtaaaacccctgaaaggcaggaaaatactttttgccaacttcccggcagatgggcattttaatccattgacaggactggctgttcacttacaatggctgggttgtgatgtacgctggtacacttccaataaatatgcagacaaactgcgaagattgaatattccgcattttcctttcagaaaagctatggatatagctgacctggagaatatgtttccggagcgtgatgccattaaaggccaggtagccaaactgaagttcgacataatcaatgcttttattcttcgcgggccggaatactatgttgacctgcaggagatacataaaagttttccatttgacgtaatggtcgctgattgcgcttttacaggaattccttttgtaacagataaaatggatatacctgttgtttctgtaggtgtgttccctcttaccgaaacatcgaaagatcttcctcccgccggcctcgggattacgccttccttttctttacccggaaaatttaaacaaagcatactacggtcggtggctgacctggtcttattccgcgagtccaataaagtaatgagaaaaatgctgaccgaacatggcattgatcatctctatacaaatgtatttgacctgatggtaaaaaaatcaacgctgctattgcaaagcggaacaccgggttttgaatattaccgcagtgatctgggaaaaaatatccgtttcattggttcattattaccctaccagtcaaaaaaacaaacaactgcatggtctgatgaaagactgaacaggtatgaaaaaattgtggtggtgacacagggcactgttgaaaagaatattgaaaagatcctcgtgcccactctggaagcctttagggatacagacttattggtaatagccacaacgggtggaagtggtacagctgagttgaaaaaaagatatcctcaaggcaacctgatcatcgaagattttattccctttggcgatatcatgccttatgcggatgtatatattaccaatggaggatatggtggtgtaatgctgggtatcgaaaaccaattgccattggtagtagcgggtattcatgaagggaaaaatgagatcaatgcaaggataggatactttgaactgggaattaacctgaaaaccgaatggcctaaaccggaacagatgaaaaaagccatagatgaagtgatcggcaacaaaaaatataaagagaatataacaaaattggcaaaagaattcagcaattaccatcccaatgaactatgcgctcagtatataagcgaagtattacaaaaaacaggcaggctttatatcagcagtaaaaaggaagaagaaaagatatactaa

SEQ ID NO: 2 - DNA, 1209 bp, Bacillus sp.
  mgtB gene
atggcaaatgtactcgtaataaatttccctggggaaggtcatattaatccgactttagctattgtaagtgagttaattcagcgaggggaaacagttgtttcttattgtattgaagattatagaaagaaggttgaagcaacaggtgcggaattccgagtgtttgagaattttctctctcaaattaatattatggaacgagtaaatgaaggtgggagccctttgatgatgctatctcatatgattgaagcatcagagcgtattgttactcaaattgtagaagaaacaaaagaggaaaaatatgattatttaatatatgataatcattttccagtaggacgtattatagcaaatattttacaattaccaagcgtttcatcttgtacaacgtttgctgttaatcagtacattaattttcatgatgggcaagaatcgagacaagtagacgaaataaatccattatatcaatcttgtttagcgggaatggaaagatggaataagcactatggaatgaaatgtaatagtatgtatgatattatgaatcatcctggtgatattacgattgtatatacttcaaaagaatatcagccgcgttcagatttatatgatgaatcgtataaatttgtaggtccatcaattgctactcgaaaagaagtggggagttttcctaccgaagatttaaaaaatgaaaaagtgattttcatttctatgggaacagtttttaatgaacaacctgctttgtatgaaaaatgttttgaagcgtttaaagatgtagatgcgacagtcgtattagtcgttggtaagaagataaatacaagtcaatttgaaaatatcccgaaaaactttaagttgtataattatgtcccgcaattagaagttttacagcatgctgatgtattcgtgacacatggtggtatgaatagttcgagtgaagcgttatattacggtgttccattagttgtaattccggtaacaggagatcagccattcgttgcaaaacgattgactgaagtaggggcaggcataacacttaatcgtaacgagttaacttctgaattgttacgtgagactgtaaagaaagtaatggatgatgtgacgtttaaggaaaatagtcgtaaagtgggagagtcgcttagaaatgctggtggatatcaaagggcagttgaggaaatatttgaattaaaaatgaagccgtacgtaaagattaaatag

SEQ ID NO: 3 - DNA, 1209 bp, Bacillus sp.
  mgtC gene
atggcaaacgtactcgtaataaatttccctggagaaggtcatataaatccgactttggctattgtaagtgagttaattcggcgaggagagacagttgtttcgtattgtattgaagattatagaaagaagattgaagcaacaggtgcagaattccgagtgtttgagaatttcctctctcaaattaatattatggagcgagtaaatgaaggcgggagtcctttgacgatgctatctcatatgattgaagcatcagagcgtattgttactcaaattgtagaagaaacaaaaggggaaaagtacgattacatgatatacgataatcattttccggtaggacgtattatagccaatgctttaaaattacctagcgtttcttcttgtacaacgtttgcttttaatcaatatattacttttaacgatgaacatgaatcaagaaaagtagatgaaacgaatccattgtatcaatcttgtttagcgggaatagaaaaatggaataaacagtatggaatgaaatgtaatagtatgtatgatattatgaatcatcctggtgatattactattgtatatacttcaaaggaatatcaaccacgttcagatgtattcgatgaatcgtataagtttgtcggcccatccattgctatgcgtaaagaagtaggcagttttcctatggaagatttaaaagataaaaaattgattttcatttctatgggaacagtttttaatgaacaacctgagctatatgaaaaatgttttgaagcatttaaagatgcagaagcgacagttatattggttgttggtaagaagataaatataagtcaatttgaaaacattccgaataactttaaattgtttaattatgtgccgcaattagaagtgttacagtatgctgatgtattcgtgacacacggtggcatgaatagttcgagtgaagcactatattacggtgttccgttagttgtaattccggtaacaggagatcagcctttagttgcgaaacgagtgaatgaagtaggggctggaataaggcttaatcgtaaagaattaacttctgaattgttacgtgaagctgtagagaaagtcgcgaatgatgtaaggtttaaggaaaatagtcgtaaagttggagagtcacttcgaaatgctggtggatataatagggcagttgatgaaatattaaaaatgaaaatgaattcatactcaaaacttaaataa

SEQ ID NO: 4 - DNA, 1176 bp, Bacillus subtilis
  mgtS gene
atgaaaaagtaccatatttcgatgatcaatatcccggcatacggacatgtcaatcctacgcttgctttagtagagaagctttgtgagaaagggcaccgtgtcacgtacgcgacgactgaggagtttgcgcccgctgttcagcaagccggtggagaagcattgctctatcatacatccttgaatattgatcctaagcaaatcagggagatgatggaaaagaatgacgcgcccctcagccttttgaaagaatcactcagcattctgccgcagcttgaggagttatataaggatgatcagcctgatctgatcatctatgactttgttgcgctggctggtaaattgtttgctgaaaagctcaatgttccggtcattaagctctgttcgtcatatgcccaaaatgaatcctttcagttaggaaatgaagacatgctgaagaaaataaaagaagcagaggctgaatttaaagcctacttggagcaagagaagttgccggctgtttcatttgaacagttagctgtgccggaagcattaaatattgtctttatgccgaagtcttttcagattcagcatgagacgttcgatgaccgtttctgttttgtcggcccctctctcggagaacgaaaggaacaagaaggcctgttgattgacaaggatgatcgcccgcttatgctgatttctttgggtacggcgtttaacgcatggccggaattttacaagatgtgcatcaaggcatttcgggattcttcatggcaagtgatcatgtcggttgggaaaacgattgatccagaaagcttggaggatattcctgctaactttactattcgccaaagtgtgccgcagcttgaggtgttagagaaagctgatttgttcatctctcatggcgggatgaacagtacgatggaagcgatgaacgcaggtgtgccgcttgtcgtcattccgcaaatgtatgagcaagagctcactgcaaatcgggttgatgaattaggccttggcgtttatttgccgaaagaggaagtgactgtttccagcctgcaggaagcggttcaggctgtatccagtgatcaagagctgctcagccgcgtcaagaatatgcaaaaagatgtaaaagaagctggcggagcggagcgtgcggcagctgagattgaagcgtttatgaaaaaatccgctgtcccgcag

SEQ ID NO: 5 - DNA, 1194 bp, Bacillus sp.
  mgtT gene
atggcgcgtgttttattcattaatgctggatcagaaggacatataaatccaactttacaagttgtagatgaattgatttctcgtggtgaagaggtcgtttatttttcaatagaagctttcagggagcggattgagaagacaggtgctactgtacgaacgattgatgatcaaaaatttataaaagcgtttctatctggaggcagaaattatttacaggaaagaataaatggccttctacatacagcggatattgtaatacctagcgttttagaacaaattgaaggtgaacattttgattacataattcatgattctatgattggctgtggccatttaattgctcaaatccttaaacttccagccataaattcatgcacatcttttgcgcaggatgaaaaatcctttgagcaaatgttaggtcatctatcaaaaaatatcccagtagaaatttatgataaaatacagaatgattttcaaaacttaacgaagggaattgctgaaaaatatggtgttgaaataaaatcatcgtatgaagttttctgtaatcctgcaccccttactattgtatatacaattaaggagttccagccttttggtgatacgtttgatgaaatatataaatttgtaggaccatctatctctgcacaaatgaaaaacagagacgttgattttacttcaattgaagaaaaaagtccgatttatatttcattaggtactgtttttaatgaagcgattgacttttataaactgtgtatgaaggcctttgagaatagtgagcatacaattgttatgtctattggtagtaaaacaaaaataagtgatctaggcgaaattcctaaaaacttcattgtgaaaaactatgtaccccaaactgagctgcttacatatacgaaactatttattacacacggcgggatgaacagtgcgcatgaaggactgtataacggggttccgctcgttgtaataccgcaaagtgcagatcagccagtagtcgcaaagcaagtggagagtcttggagcaggaataaaattacaaatgcaaggattaactgcggatcaactaagtgaaagtgtagaaatggtattaaataatccgtcatttaaagaagttgctttgaatttgaagaaatctttccaaaaatcaggtggatataaggaagctgttgatgaaatttttatatttgtaggtcagtaa

SEQ ID NO: 6 - DNA, 1209 bp, Bacillus sp.
  mgtW gene
atggcaaacgtactcgtaataaatttccctggagaaggtcatataaatccgactttagctattgtaagtgagttaattcggcgaggggaaacagttgtttcgtattgtattgaagattatagaaagaagattgaagcaacaggtgcagaattccgagtgtttgagaatttcctctctcaaattaatattatggaacgagtaaatgaaggtgggagtcctttgacgatgctatctcatatgattgaagcatcagagcgtattgttactcaaattgtagaagaaacaaaaggggaaaagtacgattacttgatatacgataatcattttccagtaggacgtattatagcgaatgttttaaaattacctagcgtttcttcttgtacaacgtttgcttttaatcagtacattacttttaatgatgaacaagaatcgagacaagtagatgaaactaatccattatatcaatcttgtttagcgggaatggaaaaatggaataggcagtatggaatgaaatgtattaatatgtatgatattatgaatcatcctggtgatattactattgtatatacttcaaaggaatatcagccgcgttcagatgtattcgatgaatcgtataagtttgtcggtccatcaattgctactcgaaaagaagtagatagctttcctatggaagatttaaaagataaacaattgattttcatttctatgggaacagtttttaatgaacaacctgagttatatgaaaaatgttttgaagcgtttaaagatgtagaagcgacagtcgtattagttgttggtaagaagataaatataagtcaatttgaaaacattccgaataactttaagttgtataattatgtgccgcaattagaagtattacagtacgctgatgtattcgtgacacacggtggtatgaatagttcgagtgaagcactatattacggtgtcccgttagttgtaattccggtaacaggagatcagcctttagttgcgaaacgagtgagtgaagtaggagctggaataaggcttaatcgtaaagaattaacttctgaattgttacgtgagactgtaaagaaagtaatgtatgatgtaacgtttaaggaaaatagtcgcaaagttggagagtcacttcgaaatgctggtgggtataatagagcagttgatgaaatatttaaaatgaaactgaattcatacttaaaacttaaataa

SEQ ID NO: 7 - protein, 459 aa, Unknown
  [CDS]1..1380 from SEQ ID NO 1
MSNLFSSQTNLASVKPLKGRKILFANFPADGHFNPLTGLAVHLQWLGCDVRWYTSNKYADKLRRLNIPHFPFRKAMDIADLENMFPERDAIKGQVAKLKFDIINAFILRGPEYYVDLQEIHKSFPFDVMVADCAFTGIPFVTDKMDIPVVSVGVFPLTETSKDLPPAGLGITPSFSLPGKFKQSILRSVADLVLFRESNKVMRKMLTEHGIDHLYTNVFDLMVKKSTLLLQSGTPGFEYYRSDLGKNIRFIGSLLPYQSKKQTTAWSDERLNRYEKIVVVTQGTVEKNIEKILVPTLEAFRDTDLLVIATTGGSGTAELKKRYPQGNLIIEDFIPFGDIMPYADVYITNGGYGGVMLGIENQLPLVVAGIHEGKNEINARIGYFELGINLKTEWPKPEQMKKAIDEVIGNKKYKENITKLAKEFSNYHPNELCAQYISEVLQKTGRLYISSKKEEEKIY

SEQ ID NO: 8 - protein, 402 aa, Bacillus sp.
  [CDS]1..1209 from SEQ ID NO 2
MANVLVINFPGEGHINPTLAIVSELIQRGETVVSYCIEDYRKKVEATGAEFRVFENFLSQINIMERVNEGGSPLMMLSHMIEASERIVTQIVEETKEEKYDYLIYDNHFPVGRIIANILQLPSVSSCTTFAVNQYINFHDGQESRQVDEINPLYQSCLAGMERWNKHYGMKCNSMYDIMNHPGDITIVYTSKEYQPRSDLYDESYKFVGPSIATRKEVGSFPTEDLKNEKVIFISMGTVFNEQPALYEKCFEAFKDVDATVVLVVGKKINTSQFENIPKNFKLYNYVPQLEVLQHADVFVTHGGMNSSSEALYYGVPLVVIPVTGDQPFVAKRLTEVGAGITLNRNELTSELLRETVKKVMDDVTFKENSRKVGESLRNAGGYQRAVEEIFELKMKPYVKIK

SEQ ID NO: 9 - protein, 402 aa, Bacillus sp.
  [CDS]1..1209 from SEQ ID NO 3
MANVLVINFPGEGHINPTLAIVSELIRRGETVVSYCIEDYRKKIEATGAEFRVFENFLSQINIMERVNEGGSPLTMLSHMIEASERIVTQIVEETKGEKYDYMIYDNHFPVGRIIANALKLPSVSSCTTFAFNQYITFNDEHESRKVDETNPLYQSCLAGIEKWNKQYGMKCNSMYDIMNHPGDITIVYTSKEYQPRSDVFDESYKFVGPSIAMRKEVGSFPMEDLKDKKLIFISMGTVFNEQPELYEKCFEAFKDAEATVILVVGKKINISQFENIPNNFKLFNYVPQLEVLQYADVFVTHGGMNSSSEALYYGVPLVVIPVTGDQPLVAKRVNEVGAGIRLNRKELTSELLREAVEKVANDVRFKENSRKVGESLRNAGGYNRAVDEILKMKMNSYSKLK

SEQ ID NO: 10 - protein, 392 aa, Bacillus subtilis
  [CDS]1..1176 from SEQ ID NO 4
MKKYHISMINIPAYGHVNPTLALVEKLCEKGHRVTYATTEEFAPAVQQAGGEALLYHTSLNIDPKQIREMMEKNDAPLSLLKESLSILPQLEELYKDDQPDLIIYDFVALAGKLFAEKLNVPVIKLCSSYAQNESFQLGNEDMLKKIKEAEAEFKAYLEQEKLPAVSFEQLAVPEALNIVFMPKSFQIQHETFDDRFCFVGPSLGERKEQEGLLIDKDDRPLMLISLGTAFNAWPEFYKMCIKAFRDSSWQVIMSVGKTIDPESLEDIPANFTIRQSVPQLEVLEKADLFISHGGMNSTMEAMNAGVPLVVIPQMYEQELTANRVDELGLGVYLPKEEVTVSSLQEAVQAVSSDQELLSRVKNMQKDVKEAGGAERAAAEIEAFMKKSAVPQ

SEQ ID NO: 11 - protein, 397 aa, Bacillus sp.
  [CDS]1..1194 from SEQ ID NO 5
MARVLFINAGSEGHINPTLQVVDELISRGEEVVYFSIEAFRERIEKTGATVRTIDDQKFIKAFLSGGRNYLQERINGLLHTADIVIPSVLEQIEGEHFDYIIHDSMIGCGHLIAQILKLPAINSCTSFAQDEKSFEQMLGHLSKNIPVEIYDKIQNDFQNLTKGIAEKYGVEIKSSYEVFCNPAPLTIVYTIKEFQPFGDTFDEIYKFVGPSISAQMKNRDVDFTSIEEKSPIYISLGTVFNEAIDFYKLCMKAFENSEHTIVMSIGSKTKISDLGEIPKNFIVKNYVPQTELLTYTKLFITHGGMNSAHEGLYNGVPLVVIPQSADQPVVAKQVESLGAGIKLQMQGLTADQLSESVEMVLNNPSFKEVALNLKKSFQKSGGYKEAVDEIFIFVGQ

SEQ ID NO: 12 - protein, 402 aa, Bacillus sp.
  [CDS]1..1209 from SEQ ID NO 6
MANVLVINFPGEGHINPTLAIVSELIRRGETVVSYCIEDYRKKIEATGAEFRVFENFLSQINIMERVNEGGSPLTMLSHMIEASERIVTQIVEETKGEKYDYLIYDNHFPVGRIIANVLKLPSVSSCTTFAFNQYITFNDEQESRQVDETNPLYQSCLAGMEKWNRQYGMKCINMYDIMNHPGDITIVYTSKEYQPRSDVFDESYKFVGPSIATRKEVDSFPMEDLKDKQLIFISMGTVFNEQPELYEKCFEAFKDVEATVVLVVGKKINISQFENIPNNFKLYNYVPQLEVLQYADVFVTHGGMNSSSEALYYGVPLVVIPVTGDQPLVAKRVSEVGAGIRLNRKELTSELLRETVKKVMYDVTFKENSRKVGESLRNAGGYNRAVDEIFKMKLNSYLKLK

SEQ ID NO: 13 - DNA, 20 bp, Artificial Sequence
  Primer cfn_GT-1for
ttatgtcccgcaattagaag

SEQ ID NO: 14 - DNA, 19 bp, Artificial Sequence
  Primer cfn_GT-for
agaaggttgaagcaacagg

SEQ ID NO: 15 - DNA, 29 bp, Artificial Sequence
  Primer cfn_GT-rev
cctactggaaaatgattatcatatattac

SEQ ID NO: 16 - DNA, 29 bp, Artificial Sequence
  Primer gtf-Nde-for
catatgagtaatttattttcttcacaaac

SEQ ID NO: 17 - DNA, 27 bp, Artificial Sequence
  Primer gtf-Bam-rev
ggatccttagtatatcttttcttcttc

SEQ ID NO: 18 - DNA, 22 bp, Artificial Sequence
  Primer mgt-1-XhoI-for
ctcgagatggcaaatgtactcg

SEQ ID NO: 19 - DNA, 25 bp, Artificial Sequence
  Primer mgt-1-XhoI-rev
ctcgagtttaatctttacgtacggc

SEQ ID NO: 20 - DNA, 17 bp, Artificial Sequence
  T3 promoter
attaaccctcactaaag

SEQ ID NO: 21 - DNA, 19 bp, Artificial Sequence
  T7 promoter
taatacgactcactatagg

SEQ ID NO: 22 - DNA, 19 bp, Artificial Sequence
  T7 terminator
gctagttattgctcagcgg

(19) United States
(12) Patent Application Publication
(10) Pub. No.: US 2016/0090578 A1
(43) Pub. Date: Mar. 31, 2016

(54) ENZYMES CATALYZING THE GLYCOSYLATION OF POLYPHENOLS

(71) Applicant: UNIVERSITÄT HAMBURG, Hamburg (DE)

(72) Inventors: Ulrich Rabausch, Hamburg (DE); Wolfgang Streit, Moenkeberg (DE); Julia Juergensen, Norderstedt (DE)

(21) Appl. No.: 14/892,003

(22) PCT Filed: May 28, 2014
(86) PCT No.: PCT/EP2014/061185

§ 371 Date: Nov. 18, 2015

(30) Foreign Application Priority Data
May 29, 2013 (EP) 13169812.8

(51) Int. Cl. C12N 9/10; C12P 19/60

(52) U.S. Cl. CPC C12N 9/1051; C12P 19/60; C12Y 204/01

(57) ABSTRACT

Enzymes catalyzing the glycosylation of polyphenols, in particular flavonoids, benzoic acid derivatives, stilbenoids, chalconoids, chromones, and coumarin derivatives. An enzyme catalyzing the glycosylation of polyphenols, wherein the enzyme
a) includes an amino acid sequence according to one of the sequences of SEQ ID NO: 7-12, or
b) is encoded by a nucleic acid with a nucleotide sequence of one of the sequences of SEQ ID NO: 1-6, or
c) is homologous to one of the enzymes defined in a) or b) above, or
d) is encoded by a nucleic acid hybridizing under stringent conditions with a nucleic acid complementary to a sequence with a nucleotide sequence of one of the sequences of SEQ ID NO: 1-6.

The invention relates to enzymes catalyzing the glycosylation of polyphenols, in particular flavonoids, benzoic acid derivatives, stilbenoids, chalconoids, chromones, and coumarin derivatives.

Polyphenols are secondary plant metabolites biosythesized via the Shikimic acid and phenylpropanoid pathway. They are aromatic compounds having hydroxyl groups at their ring system, or derivatives thereof. Flavonoids and benzoic acid derivatives are examples of polyphenols. Via secondary modification of the hydroxyl group(s) of the ring system a wide variety of natural derivatives of these compounds is formed. Sugar modifications frequently occur in nature, because they can have a significant impact on the solubility and the function of the compounds. Polyphenolic compounds are part of our daily nutrition in form of fruits and vegetables, and are known to have a positive influence on human health. Besides antioxidative and radical scavenging function they can act e.g. antiallergenic, antibacterial, antifungal, antiviral, antiinflammatory, analgesic, and even cancer protective (21). Because of these broad effects there is an increasing demand for polyphenols, e.g. specific flavonoids, in the cosmetic, the pharma- and nutraceutical industries (22-24). Meeting this demand a major problem arises from their limited availability. Flavonoids, for example, are exclusively produced in plants at low levels. The extraction is linked to the use of large quantities of solvents, and the chemical modification is not easily accomplished due to their rather complex structure (25).

The regio-specific modification of polyphenols such as flavonoids remains difficult as the directed chemical modification mostly fails. Thus enzymes have gained interest as they are able to mediate the regio- and stereochemical modification of polyphenols (26). In particular, research focuses on the specific glycosylation as a modification to influence water solubility and bioavailability of polyphenos such as, for example, flavonoids (27, 28). Enzymes that catalyze this reaction are glycosyltransferases (GTs). Generally, GTs mediate the transfer of sugar residues from a donor substrate to acceptor molecules. Based on their sequence similarities GTs are currently classified into 94 families (29). The GT family 1 (GT1) comprises enzymes that catalyze the glycosylation of small lipophilic molecules (30). These enzymes (EC 2.4.1.x) that use a nucleotide-activated donor belong to the UDP-glycosyltransferase (UGT) superfamily and are also referred as Leloir enzymes (31, 32). Glycosyltransferases acting on flavonoids also belong to GT1 (33). Enzymes of GT1 possess a GT-B fold structure and present an inverting reaction mechanism concerning the linkage of the transferred sugar moiety (34). EP 2 128 265 A1 describes glycosyltransferases of fungal origin, namely from the genus Trichoderma, for the glycosylation of flavonoids. EP 1 985 704 A1 discloses glycosyltransferases from rose plants, also acting on flavonoids. Up to now very few flavonoid-acting GT1s of prokaryotic origin have been identified and characterized in detail. The currently known flavonoid accepting UGTs derived from Gram-positive bacteria all belong to the macroside glycosyltransferase (MGT) subfamily and originate from Bacilli and Streptomycetes (35-37; see also EP 1 867 729 A1 and WO 2009/015268 A1). Furthermore a single flavonoid acting UGT derived from the Gram-negative Xanthomonas campestris is known (38).

Consequently, there is still a need for means for modifying polypenols like flavonoids, chromones and the like. It is therefore an object of the invention to provide such means. The object is solved by the subject-matter of the independent claims. Advantageous embodiments of the invention are specified in the dependent claims.

In a first aspect the invention provides an enzyme catalyzing the glycosylation of polyphenols such as, for example, phenolic acid derivatives, chalconoids, chromones, coumarin derivatives, flavonoids, and stilbenoids, wherein the enzyme

a) comprises an amino acid sequence according to one of the sequences of SEQ ID NO: 7-12, or
b) is encoded by a nucleic acid comprising a nucleotide sequence of one of the sequences of SEQ ID NO: 1-6, or
c) is homologous to one of the enzymes defined in a) or b) above, or
d) is encoded by a nucleic acid hybridizing under stringent conditions with a nucleic acid complementary to a sequence comprising a nucleotide sequence of one of the sequences of SEQ ID NO: 1-6.

The novel enzymes described herein, designated GtfC, MgtB, MgtC, MgtS, MgtT and MgtW, belong to GT family 1 and are highly active on polyphenols like flavonoids and similar molecules. The term “comprising” as used herein encompasses the term “having”, i.e. is not to be construed as meaning that further elements have necessarily to be present in an embodiment in addition to the element explicitly mentioned. For example, the term “enzyme comprising an amino acid sequence according to SEQ ID NO:X” also encompasses an enzyme having the amino acid sequence according to SEQ ID NO:X, “having” in this context meaning being exclusively composed of the amino acids in SEQ ID NO:X.

The term “homologous” as used herein in reference to a nucleic acid, protein or peptide means that a nucleic acid is in its nucleotide sequence essentially identical or similar to another nucleic acid, or a protein or peptide is in its amino acid sequence essentially identical or similar to another protein or peptide, without being completely identical to the nucleic acid or protein or peptide with which it is compared. The presence of homology between two nucleic acids or proteins or peptides can be determined by comparing a position in the first sequence with a corresponding position in the second sequence in order to determine whether identical or similar residues are present at that position. Two compared sequences are homologous to each other when a certain minimum percentage of identical or similar nucleotides or amino acids are present. Identity means that when comparing two sequences at equivalent positions the same nucleotide or amino acid is present. It may optionally be necessary to take sequence gaps into account in order to produce the best possible alignment. Similar amino acids are non-identical amino acids with the same or equivalent chemical and/or physical properties. The replacement of an amino acid with another amino acid with the same or equivalent physical and/or chemical properties is called a “conservative substitution”. Examples of physicochemical properties of an amino acid are hydrophobicity or charge. In connection with nucleic acids it is referred to a similar nucleotide or a conservative substitution when, in a coding sequence, a nucleotide within a codon is replaced with another nucleotide, the new codon, e.g. due to the degeneracy of the genetic code, still encoding the same or a similar amino acid. The skilled person knows which nucleotide or amino acid substitution is a conservative substitution. To determine the degree of similarity or identity between two nucleic acids it is preferable to take a minimum length of 60 nucleotides or base pairs, preferably a minimum length of 70, 80, 90, 100, 110, 120, 140, 160, 180, 200, 250, 300, 350 or 400 nucleotides or base pairs, or a length of at least 25%, 30%, 40%, 50%, 60%, 70%, 80%, 90%, 91%, 92%, 93%, 94%, 95%, 96%, 97%, 98%, 99%, 99.2% or 99.5% of the nucleotides in the respective nucleotide sequences. For proteins/peptides it is preferable to take a minimum length of 20, preferably a minimum length of 25, 30, 35, 40, 45, 50, 60, 80 or 100, more preferably a minimum length of 120, 140, 160, 180 or 200 amino acids, or a minimum length of 25%, 30%, 40%, 50%, 60%, 70%, 80%, 90%, 91%, 92%, 93%, 94%, 95%, 96%, 97%, 98%, 99%, 99.2% or 99.5% of the amino acids of the respective amino acid sequences compared. Particularly preferably the full length of the respective protein(s) or nucleic acid(s) is used for comparison. The degree of similarity or identity of two sequences can, for example, be determined by using the computer program BLAST (19), see, e.g. http://www.ncbi.nlm.nih.gov/BLAST/) using standard parameters. A determination of homology is dependent on the length of the sequences being compared. For the purposes of the present invention two nucleic acids, the shorter of which comprises at least 100 nucleotides, will be considered homologous when at least 70%, at least 75%, at least 80%, at least 85%, at least 90%, at least 95%, at least 96%, at least 97%, at least 98%, at least 99%, at least 99.2% or 99.5% of the nucleotides are identical and/or similar (“identities” or “positives” according to BLAST), preferably identical. In case of a sequence length of 50-99 nucleotides two nucleic acids are considered homologous when at least 80%, preferably at least 85%, 86%, 87%, 88%, 89%, or 90% of the nucleotides are identical and/or similar. In case of a sequence length of 15-49 nucleotides two nucleic acids are considered homologous when at least 90%, preferably at least 95%, 96%, 97%, 98%, 99%, 99.2% or 99.5% of the nucleotides are identical and/or similar. In the case of nucleic acids coding for a protein or peptide homology is assumed to exist if the translated amino acid sequences are homologous. As similar amino acids especially those non-identical amino acids are considered, which, on the basis of the computer program “Basic Local Alignment Search Tool”, abbreviated as BLAST (19); see e.g. http://www.ncbi.nlm.nih.gov/BLAST/) using the BLOSUM62 substitution matrix (Henikoff, S. and Henikoff, J Amino acid substitution matrices from protein blocks. Proc Natl. Acad. Sci. USA 89: 10915-10919, 1992) are designated as “positive”, i.e. have a positive score in the BLOSUM62 substitution matrix. For the purposes of the present invention, it is assumed that a homology between two amino acid sequences is present if at least 55%, preferably at least 60%, at least 65%, at least 70%, at least 75%, at least 80%, at least 85%, at least 90%, at least 95%, at least 97%, at least 98%, at least 99%, at least 99.2% or at least 99.5% of the amino acids are identical or similar, preferably identical. In particular, a homology between two sequences is assumed to exist, when, using the computer program BLAST (19); see, e.g. http://www.ncbi.nlm.nih.gov/BLAST/) using standard parameters and the BLOSUM62 substitution matrix (20) an identity or similarity (“positives”), preferably identity, of at least 55%, preferably at least 60%, at least 65%, at least 70%, at least 75%, at least 80%, at least 85%, at least 90%, at least 95%, at least 97%, at least 98%, at least 99%, at least 99.2% or at least 99.5% is obtained. The skilled person, using his expert knowledge, will readily determine which of the available BLAST programs, eg BLASTp or PLASTn, is suitable for determination of homology. In addition, the skilled person is aware of further programs for assessing homology, which he may use if necessary. Such programs are, for example, available on the website of the European Bioinformatics Institute (EMBL) (see, e.g http://www.ebi.ac.uk/Tools/similarity.html). Where such terms like “x % homologous to” or “homology of x %” are used herein, this is to be construed as meaning that two proteins or nucleic acids are considered homologous and have a sequence similarity or identity, preferably identity, of x %, e.g. 80%.

The term “hybridization” is used herein in reference to the pairing of complementary nucleic acids. Hybridization and the strength of hybridization (i.e., the strength of the association between the nucleic acids) is influenced by such factors as the degree of complementary between the nucleic acids, stringency of the conditions involved, the Tm (“melting temperature”) of a nucleic acid of the formed hybrid, and the G:C ratio within the nucleic acids.

The term “hybridizing under stringent conditions” refers to conditions of high stringency, i.e. in term of temperature, ionic strength, and the presence of other compounds such as organic solvents, under which nucleic acid hybridizations are conducted. With “high stringency” conditions, nucleic acid base pairing will occur only between nucleic acids having a high frequency of complementary base sequences. Stringent hybridization conditions are known to the skilled person (see e.g. Green M. R., Sambrook, J., Molecular Cloning: A Laboratory Manual, Cold Spring Harbor Laboratory Press; 4th edition, 2012). An example for stringent hybridization conditions is hybridizing at 42° C. in a solution consisting of 5×SSPE (43.8 g/1 NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

The term “glycosylation” relates to a reaction in which a carbohydrate as a glycosyl donor is attached to a hydroxyl or other functional group of another molecule (a glycosyl acceptor).

The term “glycosyl donor” relates to a carbohydrate, e.g. a mono- or oligosaccharide, reacting with a suitable acceptor compound to form a new glycosidic bond.

The term “carbohydrate” comprises hydrates of carbon, i.e. a compound having the stoichiometric formula Cₙ(H₂O)ₙ. The generic term includes monosaccharides, oligosaccharides and polysaccharides as well as substances derived from monosaccharides by reduction of the carbonyl group (alditols), by oxidation of one or more terminal groups to carboxylic acids, or by replacement of one or more hydroxy group(s) by a hydrogen atom, an amino group, thiol group or similar groups. It also includes derivatives of these compounds. See IUPAC. Compendium of Chemical Terminology, 2nd ed. (the “Gold Book”). Compiled by A. D. McNaught and A. Wilkinson. Blackwell Scientific Publications, Oxford (1997). XML on-line corrected version: http://goldbook.iupac.org (2006-) created by M. Nic, J. Jirat, B. Kosata; updates compiled by A. Jenkins. ISBN 0-9678550-9-8. doi:10.1351/goldbook. Last update: 2014-02-24; version: 2.3.3; doi:10.1351/goldbook.000820.

The term “polyphenols” relates to secondary plant metabolites which are biosynthesized via the Shikimic acid and phenylpropanoid pathway, and which are aromatic compounds having one, two or more hydroxyl groups directly bound to their ring system, or derivatives thereof. Examples for polyphenols are flavonoids, benzoic acid derivatives, stilbenoids, chalcones, chromones, and coumarin derivatives.

The term “flavonoid” relates to a group of compounds comprising flavones, derived from 2-phenylchromen-4-one (2-phenyl-1,4-benzopyrone) (e.g. quercetin, rutin), isoflavonoids, derived from 3-phenylchromen-4-one (3-phenyl-1,4-benzopyrone), and neoflavonoids, derived from 4-phenylcoumarine (4-phenyl-1,2-benzopyrone). The term comprises e.g flavones (e.g. luteolin, apigenin), flavanones (e.g. hesperetin, naringenin, eriodictyol), flavonols (e.g. morin, quercetin, rutin, kaempferol, myricetin, isorhamnetin, fisetin), flavanols (e.g. catechin, gallocatechin, epicatechin, epigallocatechingallat), flavanonols (e.g. taxifolin), chalcones (chalcone derivatives, e.g. isoliquiritigenin, phloretin, xanthohumol), isoflavones (e.g. genistein, daidzein, licoricidin), chromones, i.e. derivatives of chromone (1,4-benzopyrone, chromen-4-one), in particular hydroxylated chromone derivatives (e.g. noreugenin), anthocyanidins (e.g. cyanidin, delphinidin, malvidin, pelargonidin, peonidin, petunidin), and aurones (e.g. aureusidin), and acylated, glycosylated, methoxylated, and sulfoylated derivatives of the afore-mentioned compound classes. See also: IUPAC. Compendium of Chemical Terminology, 2nd ed. (the “Gold Book”). Compiled by A. D. McNaught and A. Wilkinson. Blackwell Scientific Publications, Oxford (1997). XML on-line corrected version: http://goldbook.iupac.org (2006-) created by M. Nic, J. Jirat, B. Kosata; updates compiled by A. Jenkins. ISBN 0-9678550-9-8. doi:10.1351/goldbook. Last update: 2014-02-24; version: 2.3.3; doi:10.1351/goldbook.F02424.

The term “stilbenoids” relates to hydroxylated derivatives of stilbene, and derivatives thereof, an examples being resveratrol.

The term “coumarins” relates to derivatives, in particular hydroxylated derivatives of coumarin (2H-chromen-2-one, 1-benzopyran-2-one), e.g. 7-hydroxy-4-methylcoumarin (4-MU, 4-methylumbelliferone). See also: IUPAC. Compendium of Chemical Terminology, 2nd ed. (the “Gold Book”). Compiled by A. D. McNaught and A. Wilkinson. Blackwell Scientific Publications, Oxford (1997). XML on-line corrected version: http://goldbook.iupac.org (2006-) created by M. Nic, J. Jirat, B. Kosata; updates compiled by A. Jenkins. ISBN 0-9678550-9-8. doi:10.1351/goldbook. Last update: 2014-02-24; version: 2.3.3; doi:10.1351/goldbook.001369.

The term “benzoic acid derivatives” relates to derivatives, in particular hydroxylated derivatives of benzoic acid.

In a preferred embodiment an enzyme being homologous to the enzyme of the invention is at least 75%, more preferably at least 80% or 85%, most preferred at least 90%, 95%, 96%, 97%, 98%, 99%, 99.2% or at least 99.5% homologous to the enzyme defined in a) or b) above.

In a particular preferred embodiment the enzyme of the invention comprises or has the amino acid sequence according to SEQ ID NO: 7 or is encoded by a nucleic acid comprising or having the nucleotide sequence according to SEQ ID NO: 1, or is an enzyme being at least 75%, more preferably at least 80% or 85%, most preferred at least 90%, 95%, 96%, 97%, 98% or at least 99% homologous to the enzyme comprising or having the amino acid sequence according to SEQ ID NO: 7 or being encoded by a nucleic acid comprising or having the nucleotide sequence according to SEQ ID NO: 1.

In a second aspect the invention also relates to fragments of an enzyme according to the first aspect, wherein the fragment comprises at least 60, preferably at least 65, 70, 75, 80, 85, 90, 100, 110 or at least 120 consecutive amino acids of said enzyme, and wherein the fragment catalyzes the glycosylation of a polyphenol.

In a third aspect the invention relates to a nucleic acid encoding an enzyme according to the first aspect of the invention or a fragment according to the second aspect of the invention. Preferably, the nucleic acid

a) comprises one of the nucleotide sequences according to SEQ ID NO: 1-6, or a fragment thereof, or
b) is homologous to one of the nucleic acids defined in a) above, or
c) hybridizes under stringent conditions with a nucleic acid complementary to the nucleic acid defined in a) above.

In case the invention relates to a fragment of a nucleic acid or a fragment of an enzyme it is understood that the fragment, in case of nucleic acid, encodes a peptide catalyzing the glycosylation of a polyphenol or, in case of a peptide, catalyses the glycosylation of a polyphenol.

The nucleic acid, or a fragment thereof, may be incorporated in a vector such as a plasmid as a means to introduce the nucleic acid into a host cell, e.g. a fungal, bacterial or plant cell. The nucleic acid may be functionally linked to a suitable promoter and/or other regulatory sequence(s) in order to achieve expression of the nucleic acid, or fragment, in the cell. A broad variety of suitable vectors and methods for introducing such vectors into a host cell is known to the skilled person.

In a fourth aspect the invention relates to the use of an enzyme according to the first aspect or the use of a fragment according to the second aspect of the invention for the glycosylation of polyphenols, preferably phenolic acid derivatives, flavonoids, benzoic acid derivatives, stilbenoids, chalconoids, chromones, and coumarin derivatives.

In a fifth aspect the invention relates to a method for preparing a glycoside of a polyphenol, preferably a flavonoid, benzoic acid derivative, stilbenoid, chalconoid, chromone, or coumarin derivative, comprising the step of reacting the polyphenol and a glycosyl donor with an enzyme according to one of claims 1 to 3 or a fragment thereof according to claim 4, under suitable conditions for an enzymatic reaction to occur transferring the glycosyl donor to a hydroxyl group or other functional group of the polyphenol.

Suitable conditions for an enzymatic glycosylation reaction are well known to the skilled person, and can also be derived from the following examples and prior art documents referenced herein.

The invention is now described for illustrative purposes only by means of the following examples.

### Bacterial Strains, Plasmids and Chemical Reagents

Bacterial strains and plasmids used in the present work are listed in TABLE S1 and primers are listed in TABLE S2 below.

TABLE S1
Bacterial strains, vectors and constructs used

| Designation | Genotype | Reference/Source |
|---|---|---|
| Bacillus sp. HH1500 | Bacillus cereus group soil isolate, wild type | |
| E. coli BL21 (DE3) | F− ompT dcm lon hsdS(rB− mB−) | (73), Merck KGaA, |
| | ΔgalM-ybhJ λ(DE3) | Darmstadt, Germany |
| E. coli DH5α | F− φ80 lacZΔM15 Δ(lacZYA-argF)U169 | Life Technologies, |
| | recA1 endA1 hsdR17(rk−, mk+) phoA | Frankfurt, Germany |
| | supE44 λ− thi-1 gyrA96 relA1 | |
| E. coli EPI300TM-T1^{R} | F− mcrAD (mrr-hsdRMS-mcrBC) | Epicentre, Madison, |
| | Φ80dlacZΔM15 ΔlacX74 recA1 endA1 | WI, USA |
| | araD139 Δ(ara, leu)7697 galU galK λ− rpsL | |
| | nupG trfA tonA dhfr | |
| pBluescript II SK (+) | 3.0 kb phagemid vector, lacZ, bla, PT7, PT3 | Stratagene, LaJolla, |
| | | CA, USA |
| pCC1FosTM | 8.1 kb fosmid cloning vector, CmR, lacZ, | Epicentre, Madison, |
| | PT7, repE, redF, parA, parB, parC, loxP | WI, USA |
| pDrive | 3.85 kb TA-cloning vector, lacZ, bla, KanR, | Qiagen, Hilden, |
| | PT7, PSP6 | Germany |
| pDgtfC | 5.2 kb construct of pDrive and gtfC derived | |
| | by PCR from pFOS144C11 using primer | |
| | pair gtf-Nde-for and gtf-Bam-rev (TABLE S2) | |
| pDmgtB | 5.1 kb construct of pDrive and mgtB derived | |
| | by PCR from pFOS4B2 using primer pair | |
| | mgt-1-XhoI-for and mgt-1-XhoI-rev | |
| | (TABLE S2) | |
| pET19b | 5.7 kb, overexpression vector, PT7, lacI, bla | Merck KGaA, |
| | | Darmstadt, Germany |
| pET19gtfC | 7.1 kb construct of pET19b::gtfC using NdeI | |
| | and BamHI sites | |
| pET19mgtB | 6.9 kb construct of pET19b::mgtB using | |
| | XhoI site | |
| pFOS4B2 | 46 kb fosmid from the B.sp.HH1500 library | |
| | conferring glycosyltransferase activity | |
| pFOS19G2 | 45 kb fosmid derived from the B.sp.HH1500 | |
| | library conferring glycosyltransferase | |
| | activity | |
| pFOS144C11 | 40 kb fosmid from the Elbe river sediment | |
| | metagenome library conferring | |
| | glycosyltransferase activity | |
| pSK4B2 | 6.2 kb HindIII-subclone of pFOS4B2 in | |
| | pBluescript II SK(+) | |
| pSK144C11 | 11.5 kb HindIII-subclone of pFOS144C11 in | |
| | pBluescript II SK (+) | |
| pTZ19R-Cm | 3.1 kb, pTZ19R Δbla(CmR), PT7, lacZ | (74) |
| pTZ144E1 | 4.0 kb EcoRI-subclone of pSK144C11 in | |
| | pTZ19R-Cm | |
| pTZ144E3 | 4.6 kb EcoRI-subclone of pSK144C11 in | |
| | pTZ19R-Cm | |
| pTZ144P1 | 5.7 kb PstI-subclone of pSK144C11 in | |
| | pTZ19R-Cm | |
| pTZ144P2 | 3.9 kb PstI-subclone of pSK144C11 in | |
| | pTZ19R-Cm | |

TABLE S2
Oligonucleotides and primers used for gene amplification and sequence analysis.
Recognition sites of restriction endonucleases are underlined (ID = SEQ ID NO:.

| ID: | Primer | Sequence (5′-3′) | Tm [° C.] | GC (%) |
|---|---|---|---|---|
| 13 | cfn_GT-1for | TTATGTCCCGCAATTAGAAG | 53.2 | 40 |
| 14 | cfn_GT-for | AGAAGGTTGAAGCAACAGG | 54.5 | 47.4 |
| 15 | cfn_GT-rev | CCTACTGGAAAATGATTATCATATATTAC | 58.2 | 27.6 |
| 16 | gtf-Nde-for | CATATGAGTAATTTATTTTCTTCACAAAC | 56.8 | 24.1 |
| 17 | gtf-Bam-rev | GGATCCTTAGTATATCTTTTCTTCTTC | 58.9 | 33.3 |
| 18 | mgt-1-XhoI-for | CTCGAGATGGCAAATGTACTCG | 60.4 | 50 |
| 19 | mgt-1-XhoI-rev | CTCGAGTTTAATCTTTACGTACGGC | 61.3 | 44 |
| 20 | T3 promoter | ATTAACCCTCACTAAAG | 50.0 | 42.1 |
| 21 | T7 promoter | TAATACGACTCACTATAGG | 53.3 | 36.8 |
| 22 | T7 terminator | GCTAGTTATTGCTCAGCGG | 60.2 | 52.6 |

If not otherwise stated Escherichia coli was grown at 37° C. in LB medium (1% tryptone, 0.5% yeast extract, 0.5% NaCl) supplemented with appropriate antibiotics. Bacillus isolates were grown at 30° C. in the same medium. All used chemical reagents were of analytical laboratorial grade. Polyphenolic substances were purchased from the following companies located in Germany: Merck KGaA, Darmstadt; Carl Roth GmbH, Karlsruhe; Sigma-Aldrich, Heidelberg and Applichem GmbH, Darmstadt. Additional flavonoids were ordered from Extrasynthese (Lyon, France). Stock solutions of the polyphenols were prepared in DMSO in concentrations of 100 mM.

### Isolation of DNA and Fosmid Library Construction

Strain Bacillus sp. HH1500 was originally isolated from a soil sample of the botanical garden of the University of Hamburg. DNA from Bacillus sp. HH1500 was isolated using the peqGOLD Bacterial DNA Kit (PEQLAB Biotechnologie GmbH, Erlangen, Germany) following the manufacturer protocol. The sample for the construction of the elephant feces library was derived from the Hagenbeck Zoo (Hamburg, Germany). Fresh feces of a healthy six year old female Asian elephant (Elephas maximus) named Kandy were taken and stored at −20° C. in TE buffer (10 mM TRIS-HCl, 1 mM EDTA, pH 8) containing 30% (v/v) glycerol until DNA extraction. For DNA extraction the QIAamp DNA Stool Mini Kit (Qiagen, Hilden, Germany) was used. The kit was applied according the manufacturer protocol. As recommended the incubation temperature in ASL buffer was increased to 95° C. Isolation of DNA from Elbe river sediment was performed with sediment samples from the tidal flat zone of the river Elbe nearby Glückstadt (Germany) at low tide (53° 44′40″ N, 009°, 26′14″ E). Environmental DNA was extracted using the SDS-based DNA extraction method published by Zhou and coworkers (39).

Construction of the genomic and metagenomic libraries in E. coli EPI300 cells harboring fosmid pCC1FOS was achieved with the CopyControl™ Fosmid Library Production Kit (Epicentre Biotechnologies, Madison, USA) according to the manufacturer protocol using minor modifications as previously published (40). Clones were transferred into 96 well microtitre plates containing 150 μL liquid LB medium with 12.5 μg/mL of chloramphenicol and allowed to grow overnight. Libraries were stored at −70° C. after adding 100 μL of 86% glycerol to each microtitre well. The genomic fosmid library of Bacillus sp. HH1500 comprised 1,920 clones; a total of 35,000 clones were obtained for the river Elbe sediment library and the elephant feces library encompassed a total of 20,000 clones. All libraries contained fosmids with average insert sizes of 35 kb.

### Molecular Cloning Strategies

Fragments of pCC1FOS fosmids were subcloned into pBluescript II SK+ vector using HindIII according to the restriction of the fosmid clones pFOS4B2 and pFOS144C11. The resulting plasmids were designated pSK4B2 and pSK144C11, respectively. Further subcloning of pSK144C11-derived fragments was achieved in pTZ19R-Cm with restriction enzymes EcoRI and PstI. The obtained clones were designated as pTZ144E and pTZ144P, respectively. E. coli DH5α was transformed with the plasmids by heat shock and the plasmid carrying subclones were identified by blue white screening on LB agar plates containing 10 μM 5-bromo-4-chloro-indolyl-β-D-galactopyranoside (X-Gal) and 400 μM isopropyl-β-D-thiogalactopyranoside (IPTG) after overnight growth. Different clones were analyzed by plasmid purification, followed by enzymatic digestion and agarose gel electrophoresis and/or DNA sequencing.

PCR Amplification of open reading frames (ORFs) was performed with fosmid DNA as a template. The reactions were performed in 30 cycles. To amplify mgtB the primers mgt1-XhoI-for and mgt1-XhoI-rev were used, inserting an XhoI endonuclease restriction sites 5′ and 3′ of the ORF (see TABLE S2). For cloning of gtfC primer pair gtf-Nde-for and gtf-Bam-rev was used, inserting an NdeI site including the start codon 5′ and a BamHI site 3′ of the ORF (TABLE S2). PCR fragments were ligated into pDrive using the QIAGEN PCR Cloning Kit (Qiagen, Hilden, Germany) and cloned into E. coli DH5α. Resulting clones designated as pDmgtB and pDgtfC, respectively, were analyzed for activity in biotransformation and by DNA sequencing for the correct insert. Ligation of mgtB and gtfC into expression vector pET19b (Novagen, Darmstadt, Germany) was achieved using the inserted endonuclease restriction sites of each ORF. Plasmids containing the correct insert were designated pET19mgtB and pET19gtfC, respectively. E. coli DH5α clones harboring the desired plasmids were detected by direct colony PCR using T7 terminator primer and mgt1-XhoI-for to confirm mgtB and T7 terminator primer and gtf-Nde-for to verify gtfC, respectively. Additionally, the inserts of pET19mgtB and pET19gtfC were sequenced using T7 promotor and T7 terminator primers (TABLE S2) to verify the constructs.

### Overproduction and Purification of Enzymes

For overproduction of deca-histidin (His₁₀-) tagged proteins E. coli BL21 (DE3) was transformed with pET19b constructs. An overnight preculture was harvested by centrifugation and 1% was used to inoculate an expression culture. Cells carrying pET19mgtB were grown at 22° C. until 0.7 OD₆₀₀. The culture was transferred to 17° C. and induced by 100 μM IPTG. After 16 h, the culture was harvested by centrifugation at 7.500 g at 4° C. Cells were resuspended in 50 mM phosphate buffer saline (PBS) with 0.3 M NaCl at pH 7.4 and disrupted by ultrasonication with a S2 sonotrode in a UP200S (Hielscher, Teltow, Germany) at a cycle of 0.5 and an amplitude of 75%.

The overproduction of deca-histidin-tagged GtfC was induced at 37° C. at an OD₆₀₀ of 0.6, with 100 μM IPTG. Cells were then incubated f for four hours, harvested and lysed as stated above for MgtB.

Crude cell extracts were centrifuged at 15.000 g and 4° C. to sediment the cell debris. The clarified extracts were loaded on 1 mL HisTrap FF Crude columns using the ÄKTAprime plus system (GE Healthcare). The enzymes were purified according to the manufacturer protocol for gradient elution of His-tagged proteins. Eluted protein solutions were dialyzed twice against 1,000 vol. 50 mM PBS pH 7.4 with 0.3 M NaCl at 4° C. The purification was analyzed on a 12% SDS-PAGE. The concentration of protein was determined by Bradford method using Roti-Quant (Carl Roth GmbH, Karlsruhe, Germany).

### Biotransformations and Biocatalyses

For the detection of flavonoid modifications in bacteria a biotransformation approach was used. Cultures were grown in LB medium with appropriate antibiotic overnight. Expression cultures were prepared as stated above for overproduction of enzymes. The cells were sedimented by centrifugation at 4,500 g and resuspended in 50 mM sodium phosphate buffer pH 7 supplemented with 1% (w/v) α-D-glucose. Biotransformations with a final concentration of 100 μM flavonoid inoculated from stock solutions of 100 mM in DMSO, i.e. 0.1%, were incubated in Erlenmeyer flasks at 30° C. and 175 rpm up to 24 hours. Samples of 4 mL were withdrawn and acidified with 100 μL H₃PO_{4 aq} for extraction in 2 mL ethyl acetate. They were shaken for 1 minute and phase separated by centrifugation at 2,000 g and 4° C. The supernatant was applied in TLC analysis. For quantification, samples of 100 μL were taken and dissolved 1/10 in ethyl acetate/acetic acid 3:1. These acidified ethyl acetate samples were centrifuged at 10,000 g. The supernatant was used for quantitative TLC analysis as stated below.

Fosmid clones were grown in 96 deep well plates overnight. Clones were joined in 96, 48, eight or six clones per pool. The pools were harvested by centrifugation at 4,500 g and resuspended in 50 mL LB medium containing 12.5 μg/mL chloramphenicol, CopyControl™ Autoinduction Solution (Epicentre, Madison, Wis.) (5 mM arabinose final concentration) and 100 μM of flavonoid for biotransformation. Alternatively to deep well plates, clones were precultured on agar plates. After overnight incubation the colonies where washed off with 50 mM sodium phosphate buffer pH 7, harvested by centrifugation and resuspended as outlined above. The biotransformations were incubated in 300 mL Erlenmeyer flasks at 30° C. with shaking at 175 rpm. Single clones were tested analogously but precultured in 5 mL LB and resuspended in 20 mL biotransformation media in 100 mL flasks. Samples of 4 mL were taken from the reactions after 16, 24 and 48 hours acidified with 40 μL HCl_{aq} and prepared for TLC analysis as stated above. Positive pools were verified in a second biotransformation and then systematically downsized to detect the corresponding hit in a smaller pool until the responsible single clone was identified.

Biocatalytic reactions of 1 mL contained 5 μg of purified His-tagged enzyme and were performed in 50 mM sodium phosphate buffer pH 7 at 37° C. UDP-α-D-glucose or UDP-α-D-galactose was added to final concentrations of 500 μM as donor substrate from 50 mM stock solutions in 50 mM sodium phosphate buffer pH 7. Acceptor substrates were used in concentrations of 100 μM and were added from stock solutions of 100 mM in DMSO leading to a final content of 0.1% in the reaction mixture. The reaction was stopped dissolving 100 μL reaction mixture 1/10 in ethyl acetate/acetic acid 3:1. These samples were used directly for quantitative TLC analysis.

### TLC Analyses

The supernatant transferred into HPLC flat bottom vials was used for TLC analysis. Samples of 20 μL were applied on 20×10 cm² (HP)TLC silica 60 F₂₅₄ plates (Merck KGaA, Darmstadt, Germany) versus 200 pmol of reference flavonoids. To avoid carryover of substances, i.e. prevent false positives, samples were spotted with double syringe rinsing in between by the ATS 4. The sampled TLC plates were developed in ethyl acetate/acetic acid/formic acid/water 100:11:11:27 (‘Universal Pflanzenlaufmittel’) (41). After separation the TLC plates were dried in an oven at 80° C. for five minutes. The absorbance of the separated bands was determined densitometrically depending on the absorbance maximum of the applied educt substances at 285 to 370 nm using the deuterium lamp in a TLC Scanner 3 (CAMAG, Muttenz, Switzerland). Subsequently, the substances on developed TLC plates were derivatized by dipping the plates in a methanolic solution of 1% (w/v) diphenyl boric acid β-aminoethyl ester (DPBA) (42) for one second using a Chromatogram Immersion Device (CAMAG, Muttenz, Switzerland) followed by drying the TLC plates in hot air with a fan. After two minutes the bands were visualized at 365 nm with a UV hand lamp and photographed. Alternatively, fluorescence of the bands was determined densitometrically by the TLC Scanner 3 depending on the absorbance maximum of the applied substances at 320 to 370 nm.

### Quantification of Flavonoids by TLC

To quantify flavonoids in biotransformation and biocatalytic reactions, samples were diluted 1/10 in ethyl acetate/acetic acid 3:1 to stop the reaction. Samples of 20 μL were sprayed by an ATS 4 (CAMAG, Muttenz, Switzerland) on HPTLC silica 60 F₂₅₄ plates (Merck KGaA, Darmstadt, Germany) versus different amounts of respective standard educt and product substances. TLC plates were developed, dried, derivatized and analyzed as stated above. Regression curves were calculated from the peak area of the applied reference substances to determine the amount of produced and residual flavonoids.

### HPLC-ESI-MS Analysis

HPLC was carried out on a Purospher Star RP-18e 125-4 column (Merck, Darmstadt, Germany), particle size of 3 μm, with a Rheos 2000 pump (Flux Instruments, Suisse) and set pressure limits of 0 bar minimum and a maximum of 400 bar. Injection volumes of 10 μL were separated with solvent A, water supplemented with 0.1% TFA; and solvent B, acetonitrile with 0.1% TFA in following gradient HPLC conditions: From 0 min, 0.6 mL/min 90% A, 10% B; from 14 min, 0.6 mL/min 75% A, 25% B; from 18 min, 0.6 mL/min 5% A, B=95%; from 22 min, 0.6 mL/min 5% A, 95% B; from 22.1 min, 0.6 mL/min, 90% A, 10% B; and from 28.1 min, 0.6 mL/min 90% A, 10% B. Elution was monitored with a Finnigan Surveyor PDA detector and fractions were collected by a HTC PAL autosampler (CTC Analytics). Mass spectrometry (MS) was performed on a Thermo LCQ Deca XP Plus with an ESI interface in positive ionization.

### Sequence Analysis and Genbank Entries

Automated DNA sequencing of small insert plasmids was performed using ABI377 and dye terminator chemistry following the manufacturer's instructions. Large fosmid sequences were established by 454 sequencing technology. The sequences were assembled by using Gap 4 software. ORF finding was performed with Clone manager 9 Professional software. All sequences mentioned here were deposited at GenBank, but were not published before the priority date. The DNA sequences of the Bacillus sp. HH1500 16S rRNA gene has the GenBank accession number KC145729. The fosmid derived genes from B. sp. HH1500 identified on subclone pSK4B2 are bspA (JX157885), mgtB (JX157886, SEQ ID NO: 2) and bspC (JX157887). The Elbe sediment metagenome derived fosmid subclone pSK144C11 comprised genes esmA (JX157626), gtfC (AGH18139, SEQ ID NO: 1), esmB (JX157628), and esmC (JX157629).

### Results

### Screening Method: Setup of a TLC-Based Screening Method for the Detection of Flavonoid-Modifying Enzyme Clones.

“Naturstoffreagenz A”. Since it is known that B. cereus and B. subtilis encode for glycosyltransferases mediating the glucosylation of flavonoids (36), several single bacterial isolates from the applicant's strain collections were initially tested with respect to their flavonoid modifying activities. Biotransformations using whole cells of wild type isolates confirmed the presence of flavonoid modifying enzymes in one of the strains. This strain was originally isolated from a soil sample of the botanical garden in Hamburg and was designated Bacillus sp. HH1500. Sequence analysis of a 16S rRNA gene (GenBank entry KC145729) showed a 100% identity to members of the B. cereus group (data not shown). In order to use this strain as a positive control, a fosmid library of its genomic DNA in pCC1FOS was constructed. The obtained library contained 1,920 clones with an average insert size of 35 kb. Thus, the library encompassed approximately 67 Mb of cloned gDNA hence covering the average size of a genome from B. cereus group members about ten times (43). Further, the sensitivity of the (HP)TLC-based assay was verified using a serial dilution of isoquercitrin, the 3-O-β-D-glucoside of quercetin, by spraying 10 μL of 0.78 μM up to 100 μM solutions of isoquercitrin on TLC plates and measuring the absorbance at 365 nm (TABLE 3). In addition, 10 μL of other glycosylated flavonoids were assayed at 10 μM concentrations and could be detected as clear peaks on the absorbance chromatograms (TABLE 3, and data not shown).

Based on the observed sensitivities, a systematic screening scheme was designed. Initially 96 fosmid clones were grown in deep well microtitre plates at 37° C. overnight. Cultures were then pooled and following this step, the cells were sedimented by centrifugation and resuspended in fresh LB medium containing the appropriate antibiotics and 100 μM of quercetin as acceptor substrate. After incubation for 16, 24 and 48 hours at 30° C., 4 mL samples of the pooled cultures were withdrawn and extracted with half the volume of ethyl acetate. Of these extracts 20 μL were applied on TLC silica plates and separated using ‘Universal Pflanzenlaufmittel’ as a solvent. The absorbance of the developed sample lanes was determined densitometrically at 365 nm. Additionally, bands of substrates and modified flavonoids were visualized by staining with ‘Naturstoffreagenz A’ (42), containing a 1% solution of diphenylboric acid-β-aminoethylester in methanol; and a 5% solution of polyethylengycol 4000 in ethanol (available from Carl Roth GmbH, Karlsruhe, Germany). In our hands the sensitivity of the assay was high enough to detect a single flavonoid modifying enzyme clone in a mixture of 96 clones. After the detection of a positive signal, the 96 fosmid clones was divided into pools of 48 to locate the same peak in one of the resulting two half microtitre plates. Following this procedure, the 48 clones were divided to six times eight clones and finally the eight individual clones were analyzed. This strategy was applied successfully to identify six overlapping positive clones in the Bacillus sp. HH1500 fosmid library testing all 20 microtitre plates with 1,920 clones, totally.

Of these six fosmid clones, one clone pFOS4B2 of approximately 46 kb was subcloned using the HindIII restriction site of pBluescript II SK+ vector. The obtained subclones were analyzed using the above-mentioned TLC screening technology. Thereby, a positive subclone designated pSK4B2 was identified and completely sequenced (GenBank entry JX157885-JX157887). Subclone pSK4B2 carried an insert of 3,225 bp and encoded for a gene, designated mgtB, encoding for a 402 aa protein. The identified ORF was subcloned creating plasmid pDmgtB and again assayed for activity. TLC analysis clearly confirmed the glycosylation activity of the MgtB enzyme in this construct as well. The deduced amino acid sequence of MgtB (SEQ ID NO: 8) was highly similar to a predicted B. thuringiensis macroside glycosyltransferase (TABLE 1).

TABLE 1
Open reading frames (ORF) identified on subclones pSK4B2 derived
from the active Bacillus sp. HH1500 fosmid clone and pSK144C11
derived from the river Elbe sediment active fosmid clone.

| | | | | Coverage | % Identity/ |
|---|---|---|---|---|---|
| Subclone | ORF | AA | Homolog | (%) | Similarity |
| pSK4B2 | bspA | 221 | putative protein | 100 | 99/99 |
| | | | kinase | | |
| | | | B. thuringiensis | | |
| | | | (ZP04101830) | | |
| | mgtB | 402 | macrolide | 100 | 98/99 |
| | | | glycosyltransferase | | |
| | | | B. thuringiensis | | |
| | | | (ZP04071678) | | |
| | mgtC | 261 | hypothetical | 100 | 99/100 |
| | | | membrane protein | | |
| | | | B. thuringiensis | | |
| | | | (ZP00741215) | | |
| pSK144C11 | esmA | 80 | putative UDP- | 99 | 69/80 |
| | | | NAc-muramate- | | |
| | | | L-alanin-ligase | | |
| | | | Niabella soli | | |
| | | | (ZP09632598) | | |
| | gtfC | 459 | putative UDP- | 92 | 51/71 |
| | | | glucosyltransferase | | |
| | | | Fibrisoma limi | | |
| | | | (CCH52088) | | |
| | esmB | 170 | hypothetical protein | 95 | 63/77 |
| | | | Niastella koreensis | | |
| | | | (YP005009630) | | |
| | esmC | 150 | putative membrane | 98 | 68/81 |
| | | | protein Solitalea | | |
| | | | canadensis | | |
| | | | (YP006258217) | | |

The mgtB-surrounding DNA sequences in plasmid pSK4B2 represented two truncated genes that consistently were almost identical to genes from B. thuringiensis (TABLE 1). This phylogenetic relation was in accordance to the preliminary sequence analysis of the 16S rRNA gene of Bacillus sp. HH1500 (see above).

These tests suggested that the screening procedure was suitable for the functional screening of large insert metagenome libraries. For the function-based screening of metagenomes this methodology was termed META: Metagenome Extract TLC Analysis. Although it is not fully automated high-throughput screening (HTS) technology, META allows screening of about 1,200 clones per TLC plate within a time of 48 hours for preculture, biotransformation and analysis. This number of clones appeared to be feasible if the screening was done by single person. Generally, the sampling of about one TLC plate per hour by the ATS 4 is the time limiting step of the method. But this still allows the pooled screening of several plates a day and hence throughput of numerous thousand clones a day by META.

Identification of a Novel Gylcosyltransferase from a Metagenome Library

To further apply the screening for enzyme discovery in metagenome libraries, two fosmid libraries constructed in the applicant's laboratory were tested. One library was constructed from DNA isolated from river Elbe sediment the other from isolated DNA out of fresh elephant feces. Altogether both libraries encompassed approximately 50,000 clones with an average insert size of 35 kb. Both libraries were screened using quercetin as a substrate. Using the described strategy one positive microtitre plate pool in the river Elbe-sediment-library was discovered. Further screening of this pool resulted in the identification of a single positive fosmid clone designated pFOS144C11. Biotransformations of quercetin (Q) with 48 clone pools presented one product peak (P2) by TLC separation with an Rf value comparable to that of quercitrin, the quercetin-3-O-β-L-rhamnoside. A second peak (P3) with a Rf value higher than the available reference quercetin glycones was observed in conversions with the six-clone-pool and the single fosmid clone, respectively. Clone pFOS144C11 carried a fosmid of approximately 40 kb. Subsequent restriction fragment subcloning into pBluescript II SK+ with HindIII yielded in the identification of the positive E. coli DH5α subclone pSK144C11. However, biotransformations with pSK144C11 showed two product peaks, a major one (P2) with an Rf value comparable to that of quercitrin and a minor one (P1) similar to isoquercitrin. The subclone pSK144C11 still had an insert of approximately 8.5 kb size. Further sequencing and subcloning of pSK144C11 finally identified the gene putatively responsible for the modifications which was designated gtfC. The deduced 459 amino acid sequence (see SEQ ID NO: 7) of the corresponding enzyme revealed motif similarities to UDP-glucuronosyl/UDP-glucosyltransferases. GtfC (SEQ ID NO: 7) showed a similarity of 71% to the putative glycosyltransferase of the Gram-negative bacterium Fibrisoma limi covering 92% of the protein (TABLE 1). Further cloning of the gtfC ORF into pDrive vector and biotransformation with E. coli DH5α carrying the respective construct pDgtfC confirmed the flavonoid-modifying activity of GtfC.

In summary, these results demonstrated that the developed screening procedure META is sufficiently sensitive to allow the identification of large insert clones from individual bacterial genomes (i.e. Bacillus sp. HH1500) and complex metagenome libraries (i.e. the river Elbe sediment library) showing flavonoid-modifying activities.

### Sequence Based Classification of MgtB and GtfC

To analyze the affiliation of MgtB and GtfC, a phylogenetic tree using the MEGA version 5 software (44) was calculated. The amino acid (aa) sequences of MgtB (SEQ ID NO: 8) and GtfC (SEQ ID NO: 7), and their closest sequence-based relatives determined by pBlast were aligned by ClustalW. Additionally, the sequences of the actually published prokaryotic flavonoid active GTs were aligned and finally as an outer group two eukaryotic enzymes, the flavonoid glucosyltransferase UGT85H2 from Medicago truncatula and the flavonoid rhamnosyltransferase UGT78D1 from Arabidopsis thaliana (45-46, 53). Thereof a neighbor-joining tree with 100 bootstraps was computed. As expected, MgtB from Bacillus sp. HH1500 clustered with other MGTs from the B. cereus group. At time of writing, the MGT of B. thuringiensis IBL 200 and the MGT of B. cereus G9842 turned out to be the closest relatives with an aa identity to MgtB of 98% each. Both MGTs were annotated as predicted enzymes and no substrate data were available. From the MGT cluster five other enzymes already were reported to mediate the glucosylation of flavonoids. Three of them BcGT-1 the nearest relative reported to be flavonoid active, BcGT-4, and BcGT-3 all originated from B. cereus ATCC10987 (47-49). Another flavonoid active MGT, designated BsGT-3, originates from B. subtilis strain 168 (36). The remaining flavonoid active MGT is the well-studied OleD from Streptomyces antibioticus (50, 51). GtfC was located in a distinct cluster of UGTs and appeared to be somewhat related to hypothetical enzymes from Cytophagaceae bacteria as Dyadobacter fermentans and Fibrisoma limi. Within this cluster only the UGT XcGT-2 is known to accept flavonoid substrates (38). Interestingly, rhamnosyltransferases like BSIG 4748 from Bacteroides sp. 116 and RtfA from Mycobacterium avium phylogenetically also show affiliation to this cluster but forming a separate branch.

To further characterize the identified metagenome-derived GTs, the aa residues of the C-terminal donor binding regions were compared to the motifs of the closest relatives and the known flavonoid active GTs. Here, the Rossmann fold α/β/α subdomain, the conserved donor-binding region of UGTs, is located (52). Plant UDP-glycosyltransferases like UGT85H2 and UGT78D1 exhibit a highly conserved motif in this region which is termed the (Plant Secondary Product Glycosyltransferase) PSPG motif (45, 53-54). By alignment key aa known to be of importance for NDP-sugar binding could be identified. While MgtB revealed a clear UDP-hexose binding motif consisting of highly conserved Gln289 and Glu310 residues for ribose binding and a conserved DQ, GtfC lacked this motif (45, 55, 56). Instead, GtfC presented typical residues Phe336 and Leu357 for deoxy ribose nucleotide utilization (57). Moreover the pyrophosphate binding sites in the MgtB aa sequence could be identified. However, GtfC does not possess these conserved phosphate binding residues suggesting that GtfC and related enzymes have another donor binding mode. In this context GtfC seemed to belong to a novel enzyme class underlining the low level of sequence homology.

### Overexpression and Glycosylation Patterns of MgtB and GtfC

To further characterize the novel enzymes and verify their functions, MgtB and GtfC were overexpressed and purified as His-tagged proteins in E. coli BL21 (DE3). Both genes mgtB and gtfC were ligated into the expression vector pET19b. The recombinant enzymes containing N-terminal His₁₀-tags were purified by Ni-affinity chromatography in native conditions and gradient elution. MgtB could be purified with more than 5 mg/g cell pellet (wet weight). The maximum yield of GtfC was 3 mg/g of cell pellet. The molecular weights of the proteins were verified by SDS-PAGE analysis in denaturing conditions according to Laemmli. After Coomassie-staining, His₁₀-MgtB was visible as a single band with a MW of approximately 50 kDa on a 12% SDS-PAGE. This was in accordance with the calculated molecular weight (MW) of 51.2 kDa including the N-terminal His-tag. His₁₀-GtfC revealed a MW of about 55 kDa on a 12% SDS-PAGE which was in well accordance to the calculated MW of 54.7 kDa including the N-terminal His-tag. While virtually no additional bands were visible on SDS-PAGEs with purified recombinant MgtB protein, some minor contaminating bands were still visible on the SDS-PAGE loaded with purified GtfC. In summary both proteins could be purified to allow further biochemical characterization.

The purified His₁₀-MgtB protein was able to use UDP-α-D-glucose as a donor substrate. The recombinant enzyme catalyzed the transfer of α-D-glucose residues to various polyphenols. Biocatalytic reactions were performed with 500 μM UDP-α-D-glucose as donor and 100 μM of acceptor substrate. The following flavonoids served as acceptor substrates and were modified with high yields: Luteolin, quercetin, kaempferol, tiliroside, naringenin, genistein (TABLE 2).

TABLE 2
Flavonoid substrates converted by recombinant MgtB in bioassays. Reactions of 1 mL
were carried out at 37° C. for 2 hours in triplicate with 500 μM UDP-glucose, 100 μM of the
respective flavonoid and 5 μg/mL of purified and recombinant MgtB.

| | | Conversion | Rf | |
|---|---|---|---|---|
| Substrate | | (%) | value^{a} | Product(s)^{b} |
| Quercetin | 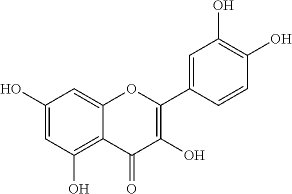 [SMILES: O=c1c(O)c(-c2ccc(O)c(O)c2)oc2cc(O)cc(O)c12] | ~100% | 0.79 0.64 0.27 0.25 | — Isoquercitrin — — |
| Kaempferol | 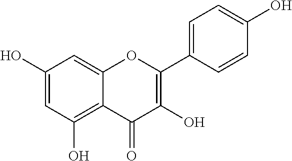 [SMILES: O=c1c(O)c(-c2ccc(O)cc2)oc2cc(O)cc(O)c12] | ~100% | 0.74 0.35 | Astragalin — |
| Luteolin | 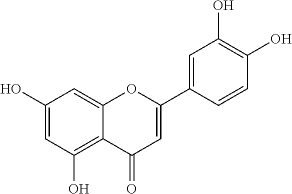 [SMILES: O=c1cc(-c2ccc(O)c(O)c2)oc2cc(O)cc(O)c12] | 82% | 0.65 0.32 | Cynaroside -3′,7-di-O-Glc |
| Naringenin | 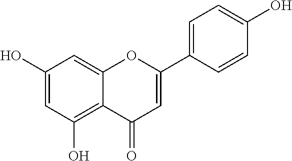 [SMILES: O=c1cc(-c2ccc(O)cc2)oc2cc(O)cc(O)c12] | 52% | 0.76 | Prunin |
| Genistein | 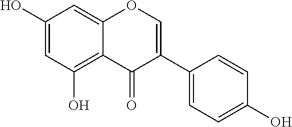 [SMILES: O=c1c(-c2ccc(O)cc2)coc2cc(O)cc(O)c12] | 72% | 0.69 | Genistin |
| Tiliroside | 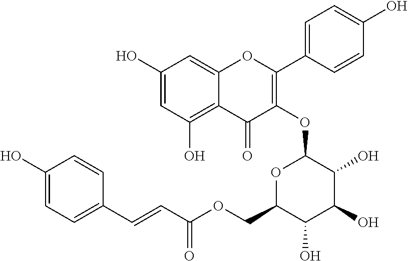 [SMILES: O=C(/C=C/c1ccc(O)cc1)OC[C@H]1O[C@@H](Oc2c(-c3ccc(O)cc3)oc3cc(O)cc(O)c3c2=O)[C@H](O)[C@@H](O)[C@@H]1O] | 83% | 0.54 | — |

^{a}Rf values and products in bold indicate the main product of the biocatalytic reactions.
^{b}Products symbolized by “—” were not specified due to unavailable reference substances.

Thereby flavonols turned out to be the best acceptor molecules. Generally, the conversion during a two-hour assay ranged from 52% for naringenin and approximately 100% for quercetin and kaempferol. Interestingly, in the presence of quercetin and kaempferol no residual educts could be monitored by HPTLC analysis. The specific educts and their observed glycones of the biocatalytic reactions are summarized in TABLE 2 together with the respective Rf values. MgtB favored the glucosylation at the C3 hydroxy group if accessible like in the aglycone flavonols quercetin and kaempferol. Further, the C7-OH was attacked and glucosylated by the enzyme which could be shown for the flavone luteolin but also the flavanone naringenin and the isoflavone genistein (TABLE 2). MgtB glucosylated luteolin also at the C3′ hydroxy group forming the 3′,7-di-O-glucoside of luteolin if the C7-OH was glucosylated previously. MgtB also catalyzed the conversion of the kaempferol derivative tiliroside, the kaempferol-3-O-6″-coumaroyl-glucoside. One glucosylated product with a Rf values of 0.54 was detected. The chalcone xanthohumol and the stilbene t-resveratrol were tested in biotransformation reactions with E. coli expressing mgtB but conversions were not quantified (data not shown). Xanthohumol yielded three detectable products whereas the biotransformation of t-resveratrol yielded one observed product by absorbance

### TLC Analysis.

Tests with recombinant and purified GtfC using UDP-α-D-glucose and UDP-α-D-galactose and quercetin as acceptor molecule suggested that dTDP-activated sugar moieties were transferred by this enzyme. This finding was confirmed by HPLC-ESI-MS analyses of biotransformation assays (see following paragraph). Unfortunately, deoxy-ribose nucleotide activated hexoses e.g. dTDP-rhamnoside were commercially not available to further analyze the obtained reaction products in more detail (58).

Biotransformations with the E. coli strain expressing GtfC and using various polyphenols as substrates yielded in conversions ranging from 52% for xanthohumol up to almost 100% turnover for most flavonols tested (TABLE 3).

TABLE 3
Flavonoid substrates and products of biotransformation assays with recombinant GtfC.
Quantification of the reaction was performed as described herein. Triplicate reactions of 50 mL
were performed in 50 mM sodium phosphate buffer (PB) pH 7.0 containing 1% (w/v) glucose and
200 μM of flavonoid at 30° C.

| | | Conversion | Rf | |
|---|---|---|---|---|
| Substrate | | (%) | value^{a} | Product(s)^{b} |
| Luteolin | 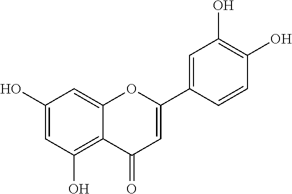 [SMILES: O=c1cc(-c2ccc(O)c(O)c2)oc2cc(O)cc(O)c12] | 86 | 0.81 0.73 0.68 0.58 | — — — — |
| Quercetin | 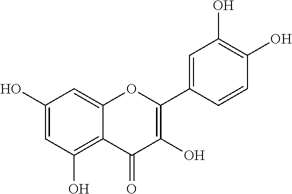 [SMILES: O=c1c(O)c(-c2ccc(O)c(O)c2)oc2cc(O)cc(O)c12] | ~100% | 0.82 0.75 0.64 | — Quercitrin Isoquercitrin |
| Kaempferol | 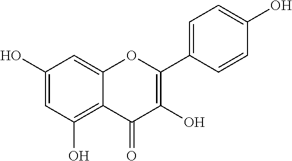 [SMILES: O=c1c(O)c(-c2ccc(O)cc2)oc2cc(O)cc(O)c12] | ~100% | 0.85 0.80 0.68 | — — Astragalin- |
| Naringenin | 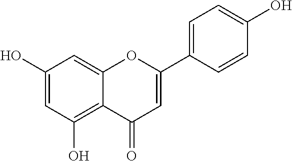 [SMILES: O=c1cc(-c2ccc(O)cc2)oc2cc(O)cc(O)c12] | 76 | 0.87 0.84 0.77 | — — Prunin |
| Genistein | 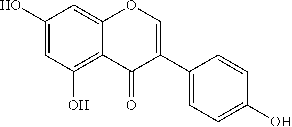 [SMILES: O=c1c(-c2ccc(O)cc2)coc2cc(O)cc(O)c12] | 68 | 0.83 0.76 0.68 | — — Genistin |
| t-Resveratrol | 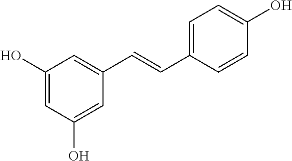 [SMILES: Oc1ccc(/C=C/c2cc(O)cc(O)c2)cc1] | 96 | 0.83 0.77 0.64 0.58 0.51 0.46 | — — — — — — |
| Xanthohumol | 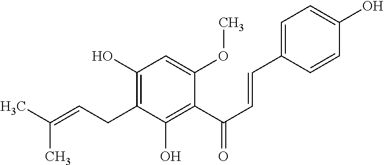 [SMILES: COc1cc(O)c(CC=C(C)C)c(O)c1C(=O)/C=C/c1ccc(O)cc1] | 52 | 0.85 0.48 | — — |

^{a}Rf values and products in bold indicate the main product of the biotransformation reactions.
^{b}Products symbolized by “—” were not specified due to unavailable reference substances

Quercetin was transformed almost completely after four-hour biotransformations and yielded three detectable products (P1-P3). To further characterize these products UV absorbance spectra were recorded and compared to the reference glycones of quercetin isoquercitrin and quercitrin (59). P1 revealed an Rf value identical to the value of isoquercitrin. Further the UV absorbance spectrum of P1 matched the spectrum of isoquercitrin. P2 revealed an Rf value identical to the one known for quercitrin. P2 also exhibited the same UV absorbance spectrum as quercitrin. P3 revealed an Rf value of 0.82, which clearly differed from the RF values of known and available quercetin glycones. Compared to isoquercitrin, P3 showed a similar hypsochromic shift of band I to a λₘₐₓ of 363 nm; however it revealed a less hypsochromic shift in band II of only 5 nm to 272 nm with a shoulder at 280 nm. It is further notable that the HPLC-ESI-MS analysis of biotransformation products of quercetin consistently identified three distinct reaction products. P1 had a RT of 17.93 min in the HPLC analysis and revealed a molecular mass of 464 u, which is equivalent to isoquercitrin. P2 revealed a RT of 18.06 min and had a molecular mass of 448 u. This mass corresponds well with the molecular mass of quercitrin. Finally, P3 with a RT of 18.31 min revealed a molecular mass of 446 u indicating the formation of a novel not further characterized quercetin glycoside.

Glycosylation patterns of GtfC on quercetin suggested a preference to act on the C3 hydroxy group mediating the transfer of different sugar residues. However, if a C3 OH-group was not available, GtfC efficiently catalyzed the glycosylation of other positions. Flavones lacking the hydroxy function at C3 were converted depending on the availability of other hydroxy groups. Pratol possessing only a single free C7-hydroxy group was converted weakly and resulted in a single detectable product. Further the biotransformation of 3′,4′-dihydroxyflavone yielded three detectable glycones and 5-methoxy-eupatorin yielded two products (data not shown); the biotransformation of the mono 4′-hydroxyflavanone yielded one glycosylated product and the glycosylation of naringenin yielded two products. The major biotransformation product of naringenin revealed the same Rf value and absorbance spectrum as prunin, the naringenin-7-O-glucoside (TABLE 3). The second naringenin glycone could not be further specified due to the lack of commercially available reference substances. Altogether these results suggested that GtfC acts on the C3, C3′, C4′ and C7 hydroxy groups of the flavonoid backbone.

In summary these data demonstrated that MgtB and GtfC possess interesting biocatalytic properties. While MgtB specifically mediated the transfer of glucose residues, GtfC transferred different hexose moieties. MgtB was capable to catalyze the glucosylation of already glycosylated flavonoids to form di-glycosides (e.g. formation of luteolin-3′,7-di-O-glucoside) and even tiliroside to generate novel glucosides not available from natural resources. In contrast, the glycosylation pattern of GtfC suggested the transfer of single sugar residues to only aglycone flavonoid forms. Interestingly, GtfC seemed to be very variable concerning its activity at various positions on the flavonoid backbone. This may lead to the formation of truly novel flavonoids naturally not available. Hence both enzymes might be helpful in the generation of new natural compounds.

Using a novel screening technology, a macroside glycosyltransferase MgtB from a soil isolate (i.e. Bacillus sp. HH1500) has been identified. A fosmid library established with DNA from this strain, which had been isolated from the local botanical garden, only recently, was initially used to develop and verify the outlined screening technology; and using the novel screening technology, MgtB was quickly identified from a pool of almost 2,000 clones. Isolation and purification of recombinant MgtB revealed a novel MGT. MgtB shared 89% aa identity with BcGT-1 from B. cereus ATCC 10987, the closest relative published to act on flavonoids. BcGT-1 was reported to catalyze the glucosylation of flavones, flavonols, flavanones and isoflavones (47). On flavonols BcGT-1 acted on C3-, C7- and C4′-hydroxy groups creating triglucosides of kaempferol (48). In contrast biocatalyses of kaempferol with MgtB yielded just two detectable glucosylated products. Instead reactions with quercetin resulted in three detectable glycones. These data suggested that MgtB acted at the C3′ OH-group. This hypothesis was also was supported by the observation that recombinant MgtB converted luteolin to luteolin-3′,7-di-O-glucoside as a byproduct. These results were in accordance with the glucosylation pattern of BcGT-3 yet another MGT from B. cereus ATCC10987 (49). Interestingly, BcGT-3 shares only 40% aa identity with MgtB but both enzymes act on the same flavonoids forming di-glucosides from flavones and flavonols at the same positions and only mono-glucosides from naringenin. The most spectacular conversion observed for MgtB was that of tiliroside. The product is likely to be the 7-O-glucoside taking the glycosylation pattern of MgtB into account. Tiliroside glycosides yet were not reported in scientific literature. This raises the possibility of the generation of new natural compounds. The natural substrates of Bacillus MGTs still have not been reported. Other MGTs like OleD usually detoxify macroside antibiotics but often possess broad acceptor tolerance (35, 60).

The metagenome-derived GtfC turned out to be a completely novel enzyme. Only seven flavonoid-active UGTs have been reported so far that originate from five different prokaryotes (35, 36, 38, 47, 49). Without XcGT-2 from Gram-negative X. campestris ATCC 33913 all remaining are MGT enzymes from Gram-positive Bacilli and Streptomycetes. MGTs play an important role in xenobiotic defense mechanisms of prokaryotes and thus show broad acceptor specificities (55, 60). This also applies for eukaryotic UGTs pointing to a biological principle of detoxification (61). To our knowledge GtfC is the first metagenome-derived GT acting on flavonoids. Moreover, it is also the first bacterial enzyme reported to transfer various dTDP activated hexose sugars to polyphenols (see below) in contrast to usually stringent donor specificities like Gtfs (57). With respect to the notion that many NDP-sugars in prokaryotes are dTDP and not UDP activated, GtfC might be a promising biocatalyst in glycodiversification approaches (58, 62, 63). GtfC is similar to predicted GTs from Cytophagaceae bacteria (64-66). These Gram-negative bacteria have large genomes suggesting extensive secondary metabolic pathways and they are well known for the presence of resistance mechanism to antibiotics as trimethoprim and vancomycin (67, 68). As commonly known glycosylation of xenobiotics is a ubiquitous detoxification process in all kingdoms of life. The phylogenetically divers members of Cytophagaceae have only recently become an object of research and a concrete estimation about the phylogenetic wideness of this family and exact taxonomic ranking still remain unclear (65, 69). Thus, the identification of the metagenome-derived GtfC and its partial characterization suggest that this group of microorganisms is perhaps highly promising resource for novel GTs and also other enzymes.

A ClustalW alignment of the donor-binding region of GtfC suggested the activated donor substrates are of deoxy-thymidine nucleoside origin. GtfC possesses the typical aa residues Phe336 for thymine base stacking and hydrophobic Leu357 for deoxy-ribose fitting (57). Concerning the donor binding of GTs GtfC appears to not exhibit the known aa residues for pyrophosphate binding. Instead of the conserved residue His/Arg in the up to date solved protein structures GtfC contains an Asn at the aa position 349 (52, 70). This applies also for the nearest GtfC relatives Dfer1940, UGT of F. limi BUZ 3 and Slin3970 as well as the NGTs RebG and BSIG4748. Further, GtfC does not show the conserved Ser/Thr residue responsible for α-phosphate binding. Instead the Gly354 appears to be of importance for the α-phosphate binding similar to the OleD transferase (55).

The assumption of dTDP activated co-substrates used by GtfC was supported by the observation that glucose, rhamnose and a third sugar residue with molecular weight of 446 were transferred by GtfC in biotransformations using intact E. coli cells. Besides, biocatalytic approaches with purified GtfC and either UDP-α-D-glucose or -galactose as donor substrates failed. In bacteria, the activated sugars, dTDP-α-D-glucose, -4-keto-6-deoxy-α-D-glucose or -4-keto-β-L-rhamnose, and -β-L-rhamnose are part of the dTDP-sugar biosynthesis pathway and are present in E. coli (71). Moreover, levels of dTDP-sugars are allosterically regulated by dTDP-rhamnose levels through activity of RmlA (72).

Four additional glycosyltransferases were identified and designated MgtT, MgtC, MgtS and MgtW.

### MgtT:

397 aa (SEQ ID NO: 11), gene 1194 bp (SEQ ID NO: 5), from Bacillus sp. BCHH1500;
99% aa identity to MGT from B. cereus B4264 (YP002367512)
Reaction in biotransformation (whole cell catalysis) with E. coli DH5α pDrive::mgtT shown for e.g. 4-Methylumbelliferone (4-MU, 7-Hydroxy-4-methylcoumarin), phloretin, homoeriodytiol, naringenin, et al.

### MgtC:

402 aa (SEQ ID NO: 9), gene 1209 bp (SEQ ID NO: 3), from Bacillus sp. BCG+1;
95% aa identity to MGT aus B. cereus ATCC10987 (NP978481)

Reaction in biotransformation (whole cell catalysis) with E. coli DH5α pDrive::mgtC shown for apigenin, luteolin; quercetin, naringenin, homoeriodytiol, phloretin, noreugenin, et al.

Exemplary reaction scheme:

### UDP-α-D-Glucose+flavonoid->Flavonoid-β-D-glucoside+UDP

### MgtS:

392 aa (SEQ ID NO: 10), gene 1179 bp (SEQ ID NO: 4), from Bacillus subtilis BSHH14
99% aa identity zu MGT YjiC aus B. subtilis (YP007533161)
Reaction in biotransformation (whole cell catalysis) with E. coli BL21(DE3) pET19b::mgtS and biocatalysis with enzyme shown for phloretin, homoeriodytiol, naringenin, apigenin, luteolin; quercetin, 4-Methylumbelliferon, noreugenin, et al.
Exemplary reaction scheme:

### UDP-α-D-Glucose+polyphenol->polyphenol-β-D-glucoside+UDP

### MgtW:

402 aa (SEQ ID NO: 12), gene 1209 bp (SEQ ID NO:6), from Bacillus sp. BCHHO3
99% aa identity to MGT from B. weihenstephanensis KBAB4 (YP001644794) Reaction in biotransformation (whole cell catalysis) with E. coli DH5α pDrive::mgtW shown for quercetin, phloretin, homoeriodyctiol, et al.

### Exemplary Reaction Scheme:

### UDP-α-D-Glucose+quercetin->quercetin 3-O-β-D-glucoside+UDP

Overview of sequences (aa=number of amino acids, bp=number of base pairs, PRT=protein, nt=number of nucleotides):

| SEQ ID NO: | Type | aa | bp/nt | description |
|---|---|---|---|---|
| 1 | DNA | | 1380 | gtfC gene |
| 2 | DNA | | 1209 | mgtB gene |
| 3 | DNA | | 1209 | mgtC gene |
| 4 | DNA | | 1179 | mgtS gene |
| 5 | DNA | | 1194 | mgtT gene |
| 6 | DNA | | 1209 | mgtW gene |
| 7 | PRT | 459 | | GtfC protein |
| 8 | PRT | 402 | | MgtB protein |
| 9 | PRT | 402 | | MgtC protein |
| 10 | PRT | 392 | | MgtS protein |
| 11 | PRT | 397 | | MgtT protein |
| 12 | PRT | 402 | | MgtW protein |
| 13 | DNA | | 20 | cfn_GT-1for |
| 14 | DNA | | 19 | cfn_GT-for |
| 15 | DNA | | 29 | cfn_GT-rev |
| 16 | DNA | | 29 | gtf-Nde-for |
| 17 | DNA | | 27 | gtf-Bam-rev |
| 18 | DNA | | 22 | mgt-1-XhoI-for |
| 19 | DNA | | 25 | mgt-1-XhoI-rev |
| 20 | DNA | | 17 | T3 promoter |
| 21 | DNA | | 19 | T7 promoter |
| 22 | DNA | | 19 | T7 terminator |

### REFERENCES

19. Altschul, S. F., Gish, W., Miller, W., Myers, E. W. & Lipman, D. J. (1990) “Basic local alignment search tool.” J Mol Biol 215:403-410
20. Henikoff, S. and Henikoff, J., Amino acid substitution matrices from protein blocks. Proc Natl Acad Sci USA 89: 10915-10919, 1992
21. Ververidis, F., Trantas, E., Douglas, C., Vollmer, G., Kretzschmar, G., and Panopoulos, N. 2007. Biotechnology of flavonoids and other phenylpropanoid-derived natural products. Part II: Reconstruction of multienzyme pathways in plants and microbes. Biotechnol J 2:1235-1249.
22. Schütz, K., Muks, E., Carle, R., and Schieber, A. 2006. Quantitative determination of phenolic compounds in artichoke-based dietary supplements and pharmaceuticals by high-performance liquid chromatography. J Agric Food Chem 54:8812-8817.
23. Leonard, E., Yan, Y., Fowler, Z. L., Li, Z., Lim, C. G., Lim, K. H., and Koffas, M. A. 2008. Strain improvement of recombinant Escherichia coli for efficient production of plant flavonoids. Mol Pharm 5:257-265.
24. Wang, C., Meek, D. J., Panchal, P., Boruvka, N., Archibald, F. S., Driscoll, B. T., and Charles, T. C. 2006. Isolation of poly-3-hydroxybutyrate metabolism genes from complex microbial communities by phenotypic complementation of bacterial mutants. Appl Environ Microbiol 72:384-391.
25. Manach, C., Scalbert, A., Morand, C., Remesy, C., and Jimenez, L. 2004. Polyphenols: food sources and bioavailability. Am J Clin Nutr 79:727-747.
26. Das, S., and Rosazza, J. P. 2006. Microbial and enzymatic transformations of flavonoids. J Nat Prod 69:499-508.
27. Graefe, E. U., Wittig, J., Mueller, S., Riethling, A. K., Uehleke, B., Drewelow, B., Pforte, H., Jacobasch, G., Derendorf, H., and Veit, M. 2001. Pharmacokinetics and bioavailability of quercetin glycosides in humans. J Clin Pharmacol 41:492-499.
28. Kren, V., and Martinkova, L. 2001. Glycosides in medicine: The role of glycosidic residue in biological activity. Curr Med Chem 8:1303-28.
29. Coutinho, P. M., Deleury, E., Davies, G. J., and Henrissat, B. 2003. An evolving hierarchical family classification for glycosyltransferases. J Mol Biol 328:307-317.
30. Bowles, D., Lim, E. K., Poppenberger, B., and Vaistij, F. E. 2006. Glycosyltransferases of lipophilic small molecules. Annu Rev Plant Biol 57:567-597.
31. Mackenzie, P. I., Owens, I. S., Burchell, B., Bock, K. W., Bairoch, A., Belanger, A., Fournel-Gigleux, S., Green, M., Hum, D. W., Iyanagi, T., Lancet, D., Louisot, P., Magdalou, J., Chowdhury, J. R., Ritter, J. K., Schachter, H., Tephly, T. R., Tipton, K. F., and Nebert, D. W. 1997. The UDP glycosyltransferase gene superfamily: recommended nomenclature update based on evolutionary divergence. Pharmacogenetics 7:255-269.
32. Lairson, L. L., Henrissat, B., Davies, G. J., and Withers, S. G. 2008. Glycosyltransferases: structures, functions, and mechanisms. Annu Rev Biochem 77:521-555.
33. Osmani, S. A., Bak, S. r., and Møller, B. L. 2009. Substrate specificity of plant UDP-dependent glycosyltransferases predicted from crystal structures and homology modeling. Phytochemistry 70:325-347.
34. Breton, C., Snajdrova, L., Jeanneau, C., Koca, J., and Imberty, A. 2006. Structures and mechanisms of glycosyltransferases. Glycobiology 16:29R-37.
35. Yang, M., Proctor, M. R., Bolam, D. N., Errey, J. C., Field, R. A., Gilbert, H. J., and Davis, B. G. 2005. Probing the breadth of macrolide glycosyltransferases: in vitro remodeling of a polyketide antibiotic creates active bacterial uptake and enhances potency. J Am Chem Soc 127:9336-9337.
36. Jeon, Y., Kim, B., Kim, J., Cheong, Y., and Alm, J.- H. 2009. Enzymatic Glycosylation of Phenolic Compounds Using BsGT-3. Journal of the Korean Society for Appl Biol Chem 52:98-101.
37. Rao, K. V., and Weisner, N. T. 1981. Microbial Transformation of Quercetin by Bacillus cereus. Appl Environ Microbiol 42:450-452.
38. Kim, H. J., Kim, B. G., Kim, J. A., Park, Y., Lee, Y. J., Lim, Y., and Ahn, J. H. 2007. Glycosylation of flavonoids with E. coli expressing glycosyltransferase from Xanthomonas campestris. J Microbiol Biotechnol 17:539-542.
39. Zhou, J., Bruns, M. A., and Tiedje, J. M. 1996. DNA recovery from soils of diverse composition. Appl Environ Microbiol 62:316-322.
40. Ilmberger, N., Meske, D., Juergensen, J., Schulte, M., Barthen, P., Rabausch, U., Angelov, A., Mientus, M., Liebl, W., Schmitz, R. A., and Streit, W. R. 2012. Metagenomic cellulases highly tolerant towards the presence of ionic liquids—linking thermostability and halotolerance. Appl Microbiol Biotechnol 95:135-146.
41. Wagner, H., Bladt, S., Zgainski, E. M. 1983. Drogenanalyse, Dünnschichtchromatographische Analyse von Arzneidrogen. Springer Publisher, Berlin Heidelberg New York.
42. Neu, R. 1957. Chelate von Diarylborsäuren mit aliphatischen Oxyalkylaminen als Reagenzien für den Nachweis von Oxyphenyl-benzo-γ-pyronen. Naturwissenschaften 44:181-182.
43. Tourasse, N. J., Helgason, E., Okstad, O. A., Hegna, I. K., and KolstØ, A. B. 2006. The Bacillus cereus group: novel aspects of population structure and genome dynamics. Appl Microbiol 101:579-593.
44. Tamura K, P. D., Peterson N, Stecher G, Nei M, and Kumar S. 2011. MEGA5: Molecular Evolutionary Genetics Analysis using Maximum Likelihood, Evolutionary Distance, and Maximum Parsimony Methods. Mol Biol Evol 28:2731-2739.
45. Li, L., Modolo, L. V., Escamilla-Trevino, L. L., Achnine, L., Dixon, R. A., and Wang, X. 2007. Crystal structure of Medicago truncatula UGT85H2—insights into the structural basis of a multifunctional (iso)flavonoid glycosyltransferase. J Mol Biol 370:951-963.
46. Jones, P., Messner, B., Nakajima, J.- I., Schaffner, A. R., and Saito, K. 2003. UGT73C6 and UGT78D1, Glycosyltransferases Involved in Flavonol Glycoside Biosynthesis in Arabidopsis thaliana. J Biol Chem 278:43910-43918.
47. Ko, J. H., Gyu Kim, B., and Joong-Hoon, A. 2006. Glycosylation of flavonoids with a glycosyltransferase from Bacillus cereus. FEMS Microbiol Lett 258:263-268.
48. Jung, N. R., Joe, E. J., Kim, B. G., Ahn, B. C., Park, J. C., Chong, Y., and Ahn, J. H. 2010. Change of Bacillus cereus flavonoid O-triglucosyltransferase into flavonoid O-monoglucosyltransferase by error-prone polymerase chain reaction. J Microbiol Biotechnol 20:1393-1396.
49. Ahn, B. C., Kim, B. G., Jeon, Y. M., Lee, E. J., Lim, Y., and Ahn, J. H. 2009. Formation of Flavone Di-O-Glucosides Using a Glycosyltransferase from Bacillus cereus. J Microbiol Biotechnol 19:387-390.
50. Choi, S. H., Ryu, M., Yoon, Y. J., Kim, D. M., and Lee, E. Y. 2012. Glycosylation of various flavonoids by recombinant oleandomycin glycosyltransferase from Streptomyces antibioticus in batch and repeated batch modes. Biotechnol Lett 34:499-505.
51. Williams, G. J., Zhang, C., and Thorson, J. S. 2007. Expanding the promiscuity of a natural-product glycosyltransferase by directed evolution. Nat Chem Biol 3:657-662.
52. Hu, Y., and Walker, S. 2002. Remarkable structural similarities between diverse glycosyltransferases. Chem Biol 9:1287-1296.
53. Hughes, J., and Hughes, M. A. 1994. Multiple secondary plant product UDP-glucose glucosyltransferase genes expressed in cassava (Manihot esculenta Crantz) cotyledons. Mitochondrial DNA 5:41-49.
54. Paquette, S., Moller, B. L., and Bak, S. 2003. On the origin of family 1 plant glycosyltransferases. Phytochemistry 62:399-413.
55. Bolam, D. N., Roberts, S., Proctor, M. R., Turkenburg, J. P., Dodson, E. J., Martinez-Fleites, C., Yang, M., Davis, B. G., Davies, G. J., and Gilbert, H. J. 2007. The crystal structure of two macrolide glycosyltransferases provides a blueprint for host cell antibiotic immunity. Proc Natl Acad Sci USA 104:5336-5341.
56. Offen, W., Martinez-Fleites, C., Yang, M., Kiat-Lim, E., Davis, B. G., Tarling, C. A., Ford, C. M., Bowles, D. J., and Davies, G. J. 2006. Structure of a flavonoid glucosyltransferase reveals the basis for plant natural product modification. Embo J 25:1396-1405.
57. Mulichak, A. M., Lu, W., Losey, H. C., Walsh, C. T., and Garavito, R. M. 2004. Crystal structure of vancosaminyltransferase GtfD from the vancomycin biosynthetic pathway: interactions with acceptor and nucleotide ligands. Biochemistry 43:5170-5180.
58. Lim, E.- K., Ashford, D. A., and Bowles, D. J. 2006. The Synthesis of Small-Molecule Rhamnosides through the Rational Design of a Whole-Cell Biocatalysis System. ChemBioChem 7:1181-1185.
59. Mabry, T. J., Markham, K. R., and Thomas, M. B. 1970. The systematic identification of flavonoids. New York. Springer-Verlag.
60. Gantt, R. W., Goff, R. D., Williams, G. J., and Thorson, J. S. 2008. Probing the aglycon promiscuity of an engineered glycosyltransferase. Angew Chem Int Ed Engl 47:8889-8892.
61. Vogt, T., and Jones, P. 2000. Glycosyltransferases in plant natural product synthesis: characterization of a supergene family. Trends Plant Sci 5:380-386.
62. Williams, G. J., Gantt, R. W., and Thorson, J. S. 2008. The impact of enzyme engineering upon natural product glycodiversification. Curr Opin Chem Biol 12:556-564.
63. Yoon, J. A., Kim, B. G., Lee, W. J., Lim, Y., Chong, Y., and Ahn, J. H. 2012. Production of a Novel Quercetin Glycoside through Metabolic Engineering of Escherichia coli. Appl Environ Microbiol 78:4256-4262.
64. Filippini, M., Kaech, A., Ziegler, U., and Bagheri, H. C. 2011. Fibrisoma limi gen. nov., sp. nov., a filamentous bacterium isolated from tidal flats. Int J Sys Evol Microbiol 61:1418-1424.
65. Lail, K., Sikorski, J., Saunders, E., Lapidus, A., Glavina Del Rio, T., Copeland, A., Tice, H., Cheng, J. F., Lucas, S., Nolan, M., Bruce, D., Goodwin, L., Pitluck, S., Ivanova, N., Mavromatis, K., Ovchinnikova, G., Pati, A., Chen, A., Palaniappan, K., Land, M., Hauser, L., Chang, Y. J., Jeffries, C. D., Chain, P., Brettin, T., Detter, J. C., Schutze, A., Rohde, M., Tindall, B. J., Goker, M., Bristow, J., Eisen, J. A., Markowitz, V., Hugenholtz, P., Kyrpides, N. C., Klenk, H. P., and Chen, F. 2010. Complete genome sequence of Spirosoma linguale type strain (1). Stand Genom Sci 2:176-185.
66. Lang, E., Lapidus, A., Chertkov, O., Brettin, T., Detter, J. C., Han, C., Copeland, A., Glavina Del Rio, T., Nolan, M., Chen, F., Lucas, S., Tice, H., Cheng, J. F., Land, M., Hauser, L., Chang, Y. J., Jeffries, C. D., Kopitz, M., Bruce, D., Goodwin, L., Pitluck, S., Ovchinnikova, G., Pati, A., Ivanova, N., Mavrommatis, K., Chen, A., Palaniappan, K., Chain, P., Bristow, J., Eisen, J. A., Markowitz, V., Hugenholtz, P., Goker, M., Rohde, M., Kyrpides, N. C., and Klenk, H. P. 2009. Complete genome sequence of Dyadobacter fermentans type strain (NS 114). Stand Genom Sci 1:133-140.
67. Chelius, M. K., and Triplett, E. W. 2000. Dyadobacter fermentans gen. nov., sp. nov., a novel gram-negative bacterium isolated from surface-sterilized Zea mays stems. IntJ Sys Evol Microbiol 50:751-758.
68. Finster, K. W., Herbert, R. A., and Lomstein, B. A. 2009. Spirosoma spitsbergense sp. nov. and Spirosoma luteum sp. nov., isolated from a high Arctic permafrost soil, and emended description of the genus Spirosoma. Int J Sys Evol Microbiol 59:839-844.
69. Filippini, M., Svercel, M., Laczko, E., Kaech, A., Ziegler, U., and Bagheri, H. C. 2011. Fibrella aestuarina gen. nov., sp. nov., a filamentous bacterium of the family Cytophagaceae isolated from a tidal flat, and emended description of the genus Rudanella Weon et al. 2008. Int J Sys Evol Microbiol 61:184-189.
70. Ha, S., Gross, B., and Walker, S. 2001. E. Coli MurG: a paradigm for a superfamily of glycosyltransferases. Curr Drug Targets Infect Disord 1:201-213.
71. Samuel, G., and Reeves, P. 2003. Biosynthesis of O-antigens: genes and pathways involved in nucleotide sugar precursor synthesis and O-antigen assembly. Carbohydr Res 338:2503-2519.
72. Giraud, M. F., and Naismith, J. H. 2000. The rhamnose pathway. Curr Opin Struct Biol 10:687-696.
73. Studier, F. W., Daegelen, P., Lenski, R. E., Maslov, S., and Kim, J. F. 2009. Understanding the Differences between Genome Sequences of Escherichia coli B Strains REL606 and BL21 (DE3) and Comparison of the E. coli B and K-12 Genomes. J Mol Biol 394:653-680.
74. Larbig, K. D., Christmann, A., Johann, A., Klockgether, J., Hartsch, T., Merkl, R., Wiehlmann, L., Fritz, H.- J., and Tümmler, B. 2002. Gene Islands Integrated into tRNAGly Genes Confer Genome Diversity on a Pseudomonas aeruginosa Clone. J Bacteriol 184:6665-6680.

1. An enzyme catalyzing the glycosylation of polyphenols, wherein the enzyme
a) comprises the amino acid sequence according to SEQ ID NO: 7, or
b) is encoded by a nucleic acid comprising the nucleotide sequence of SEQ ID NO: 1, or
c) is at least 75% homologous to one of the enzymes defined in a) or b) above, or
d) is encoded by a nucleic acid hybridizing under stringent conditions with a nucleic acid complementary to a sequence comprising the nucleotide sequence of SEQ ID NO: 1.

2. The enzyme according to 1, wherein the enzyme is at least 80% homologous to the enzyme defined in a) or b) above.

3. A fragment of an enzyme according to claim 1, wherein the fragment comprises at least 60 consecutive amino acids of said enzyme, and wherein the fragment catalyzes the glycosylation of a polyphenol.

4. A nucleic acid encoding an enzyme according to claim 1 or a fragment thereof, the fragment of nucleic acid encoding a fragment of an enzyme, wherein the fragment of enzyme catalyzes the glycosylation of a polyphenol.

5. A nucleic acid according to claim 3, wherein the nucleic acid
a) comprises the nucleotide sequence according to SEQ ID NO: 1, or a fragment thereof, the fragment encoding a fragment of an enzyme catalyzing the glycosylation of a polyphenol, or
b) is homologous to one of the nucleic acids defined in a) above, or
c) hybridizes under stringent conditions with a nucleic acid complementary to the nucleic acid defined in a) above.

6. (canceled)

7. A method for preparing a glycoside of a polyphenol, comprising the step of reacting the polyphenol and a glycosyl donor with an enzyme according to claim 1 or a fragment thereof, wherein the fragment catalyzes the glycosylation of a polyphenol, under suitable conditions for an enzymatic reaction to occur transferring the glycosyl donor to a hydroxyl group or other functional group of the polyphenol.

8. The method of claim 7, wherein the polyphenol is a phenolic acid derivative, flavonoid, benzoic acid derivative, stilbenoid, chalconoid, chromone, or coumarin derivative.

9. The enzyme according to 1, wherein the enzyme is at least 90% homologous to the enzyme defined in a) or b) above.

10. The enzyme according to 1, wherein the enzyme is at least 97% homologous to the enzyme defined in a) or b) above.

11. The enzyme according to 1, wherein the enzyme is at least 98% homologous to the enzyme defined in a) or b) above.

12. The enzyme according to 1, wherein the enzyme is at least 99% homologous to the enzyme defined in a) or b) above.

13. The enzyme according to 1, wherein the enzyme is at least 99.2% homologous to the enzyme defined in a) or b) above.

14. The enzyme according to 1, wherein the enzyme is at least 99.5% homologous to the enzyme defined in a) or b) above.

15. A fragment of an enzyme according to claim 1, wherein the fragment comprises at least 70 consecutive amino acids of said enzyme, and wherein the fragment catalyzes the glycosylation of a polyphenol.

16. A fragment of an enzyme according to claim 1, wherein the fragment comprises at least 80 consecutive amino acids of said enzyme, and wherein the fragment catalyzes the glycosylation of a polyphenol.

17. A fragment of an enzyme according to claim 1, wherein the fragment comprises at least 90 consecutive amino acids of said enzyme, and wherein the fragment catalyzes the glycosylation of a polyphenol.

18. A fragment of an enzyme according to claim 1, wherein the fragment comprises at least 100 consecutive amino acids of said enzyme, and wherein the fragment catalyzes the glycosylation of a polyphenol.

19. A fragment of an enzyme according to claim 1, wherein the fragment comprises at least 110 consecutive amino acids of said enzyme, and wherein the fragment catalyzes the glycosylation of a polyphenol.

20. A fragment of an enzyme according to claim 1, wherein the fragment comprises at least 120 consecutive amino acids of said enzyme, and wherein the fragment catalyzes the glycosylation of a polyphenol.
